(19) United States
(12) Patent Application Publication
(10) Pub. No.: US 2025/0090643 A1
(43) Pub. Date: Mar. 20, 2025

(54) FUNCTIONALIZED BIOCATALYTICAL COMPOSITIONS

(71) Applicant: PERSEO PHARMA AG, Muttenz (CH)

(72) Inventors: Emilie LAPRÉVOTTE, Hégenheim (FR); Yves Victor René DUDAL, Bottmingen (CH); Manon BRIAND, Ennetbaden (CH); Patrick SHAHGALDIAN, Hegenheim (FR)

(21) Appl. No.: 18/729,752

(22) PCT Filed: Jan. 19, 2023
(86) PCT No.: PCT/EP2023/051194

§ 371 Date: Jul. 17, 2024

(30) Foreign Application Priority Data
Jan. 20, 2022 (EP) 22152486.1

(51) Int. Cl. A61K 38/46; A61K 47/02; A61K 47/10; A61K 47/26

(52) U.S. Cl. CPC A61K 38/465; A61K 47/02; A61K 47/10; A61K 47/26

(57) ABSTRACT

The present invention relates to a composition comprising a solid carrier, a protein or a fragment thereof immobilized on the surface of the solid carrier, a protective layer to protect the protein or the fragment thereof by embedding the protein or the fragment thereof, and a functional constituent immobilized on the surface of the protective layer, wherein the functional constituent immobilized on the surface of the protective layer is a polymer comprising repeat units, wherein each repeat unit comprises at least one amino group and/or at least one thiol group.

### THE FIELD OF THE INVENTION

The present invention relates to a composition comprising a solid carrier, a protein or a fragment thereof immobilized on the surface of the solid carrier, a protective layer to protect the protein or a fragment thereof by embedding the protein or a fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group. The present invention also relates to methods of producing said composition.

### BACKGROUND OF THE INVENTION

Proteins such as enzymes are frequently needed, e.g. in industrial applications, diagnostics or for therapeutic use. In order to stabilize the proteins and/or to provide resistance to various types of stresses it has been suggested in the prior art to immobilize the proteins on the surface of a carrier and to protect them with a layer of protective material. Such an approach has been described e.g. in WO2015/014888 which discloses a biocatalytical composition comprising a solid carrier, a functional constituent like an enzyme and a protective layer for protecting the functional constituent by embedding the functional constituent and a process to produce such biocatalytical composition. However biocatalytical compositions as described e.g in WO2015/014888 cannot be used in therapeutic application due to their lack of biocompability and bioavailability. Thus there is a need to provide biocatalytical compositions compatible and useful for therapeutic applications.

### SUMMARY OF THE INVENTION

The present invention provides a composition comprising a solid carrier, a protein or a fragment thereof immobilized on the surface of the solid carrier, a protective layer to protect the protein or a fragment thereof by embedding the protein or a fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group.

The present invention provides also a method of producing said composition comprising a solid carrier, a protein or a fragment thereof immobilized on the surface of the solid carrier, a protective layer to protect the protein or a fragment thereof by embedding the protein or a fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group, the method comprising the following steps:
(a) providing a solid carrier;
(b) immobilizing a protein or a fragment thereof on the solid carrier;
(c) forming a protective layer on the surface of the solid carrier to protect the protein or a fragment thereof immobilized on the solid carrier;
(d) immobilizing a functional constituent on the surface of the protective layer, wherein the functional constituent immobilized on the surface of the protective layer is a polymer comprising repeat units wherein each repeat unit comprises at least one amino group and/or at least one thiol group.

It has been surprisingly found by the inventors of the present application that compositions as provided by the present invention if applied therapeutically have an unexpected high biodistribution specificity and surprisingly high muco adhesive properties in vitro and in vivo, show low cytotoxicity, and do not disrupt the intestinal barrier if localized in the gastrointestinal tract, thus making them extremely promising for therapeutic use, in particular for therapeutic use in enzyme replacement therapy (ERT).

### BRIEF DESCRIPTION OF THE FIGURES

FIG. 1) shows a schematic representation of the process for the production of the composition of the invention: a) a protein or a fragment thereof is immobilized on the solid carrier; b) and c) a protective layer grows around the immobilized protein or the fragment thereof embedding the immobilized protein or the fragment thereof, and d) a functional constituent is immobilized on the surface of the protective layer.

FIG. 2) shows functionalization of nanoparticles with chitosan. (A) Shielded nanoparticles (NP-1) were reacted with FITC-chitosan for 30 min, at 20° C. stirring at 400 rpm. Histogram represents the fluorescence intensity (lₑₓ: 489 nm; lₑₘ: 515 nm) of shielded (NP-1)- and reacted (NP-2)-nanoparticles. (B) Kinetics of Cu(II)-catalyzed azide-alkyne cycloaddition reaction between ethynyl-modified silica nanoparticles (SNPs) and 3-Azido-7-hydroxycoumarin. (C) Kinetics of copper-free azide-alkyne cycloaddition reaction between dibenzocyclooctyne-maleimide and 3-Azido-7-hydroxycoumarin.

FIG. 3) shows ex-vivo interaction of chitosan-functionalized-shielded-nanoparticles with mucus layer depending on the level of surface functionalization of the nanoparticles. Fluorescent-shielded-nanoparticles (NP-1) and fluorescent-partially-(NP-3) or fully-(NP-2) chitosan-functionalized-shielded-nanoparticles (500 μg/mL) were added to a layer of porcine mucus for 1 h. The binding of nanoparticles on the mucus were assessed by the measurement of fluorescence (lₑₓ: 489 nm; lₑₘ: 515 nm). Histograms represent the percentage of nanoparticles bound on the mucus.

FIG. 4) shows ex-vivo interaction of chitosan-functionalized-shielded-nanoparticles with mucus layer depending on the molecular weight (MW) of chitosan immobilized at the surface of nanoparticles. Fluorescent-chitosan-functionalized-shielded-nanoparticles (500 μg/mL) were added to a layer of porcine mucus for 1h. (A) Images show the nanoparticles (dark area within the circle) bound on the mucus layer after washing. (B) The quantification of nanoparticles bound on the mucus layer is shown in the table. The different formulations of nanoparticles assessed are: NP-4: fluorescent-human-recombinant-lipase-shielded-nanoparticles; NP-6: fluorescent-human-recombinant-lipase-shielded-nanoparticles partially functionalized with medium MW chitosan by electrostatic interactions; NP-7: fluorescent-human-recombinant-lipase-shielded-nanoparticles partially functionalized with medium MW chitosan by click chemistry, NP-8: fluorescent-human-recombinant-lipase-shielded-nanoparticles partially functionalized with low MW chitosan by click chemistry.

FIG. 5) shows ex-vivo interaction of nanoparticles functionalized with different polymer comprising amino groups with mucus layer. Fluorescent-shielded-nanoparticles (500 μg/mL) were added to a layer of porcine mucus for 1 h. (A) Images show the nanoparticle (dark area within the circle) bound on the mucus layer after washing. (B) The quantification of nanoparticles bound on the mucus layer is shown in the table. The different nanoparticles formulations are the following: NP-4: fluorescent-human-recombinant-lipase-shielded-nanoparticles; NP-9: fluorescent-human-recombinant-lipase-shielded-nanoparticles functionalized with an additional layer of polymerized APTES at the surface; NP-5: fluorescent-human-recombinant-lipase-shielded-nanoparticles fully functionalized with medium MW chitosan by electrostatic interactions, and NP-10: fluorescent-human-recombinant-lipase-shielded-nanoparticles functionalized with polymerized silane-PEG-NH2 at the surface.

FIG. 6) shows interaction of chitosan-functionalized-shielded-nanoparticles with the intestinal barrier model. (A) Differentiated Caco-2 and differentiated Caco-2/HT29-MTX-E12 co-culture were stained with Alcian blue and imaged with a ZEISS light microscope. The dark area is mucus stained with Alcian blue. Representative images are shown. (B) Fluorescent-fully-chitosan-functionalized-shielded-nanoparticles (NP-2) were added to the apical side of differentiated intestinal model barrier for 24h. Images show the nanoparticles (dark area) bound on the mucus after washing of the intestinal cells.

FIG. 7) shows biodistribution of fully-chitosan-functionalized-shielded-nanoparticles in mice. Radioactive-fully-chitosan-functionalized-shielded-nanoparticles (NP-2) were orally administrated by gavage to mice. At terminal time points, the gastrointestinal tract (A) and blood and organs (B) were collected and counted in gamma counter. Data are expressed as percentage of the injected dose per gram of organ (% ID/organ)

FIG. 8) shows in vitro cytotoxicity assessment of the nanoparticles. Caco-2 (A-B) and HT29-MTX-E12 (C-D) cells were exposed to increasing concentrations of nanoparticles (NP-1 or NP-2) for 24h (A-C) and 48h (B-D). Cell damages were assessed using the MTT (3-[4,5-dimethylthiazol-2-yl]-2,5 diphenyl tetrazolium bromide) assay. Cellular viability values are expressed as percentage of control cells (untreated cells). Triton (1 mg/mL) is used as negative control. Error bars indicate standard deviation.

FIG. 9) shows effects of nanoparticles exposure on the transepithelial electrical resistance (TEER). Differentiated Caco-2 (A-B) and differentiated Caco-2/HT29-MTX-E12 co-culture (C-D) were exposed to nanoparticles (NP-1 or NP-2), covering 100% of cell surface, for 24h (A-C) and 48h (B-D). The barrier integrity was assessed by the measurement of the TEER. “NP-1 w/o cells” and “NP-2 w/o cells” refers to the addition of the nanoparticles in the transwell system in absence of cells to evaluate the impact of the presence of the nanoparticles on the TEER measurement (background signal). EGTA (2.5 mM) is a calcium chelator that disrupts the tight junctions, it is used as control for the loss of barrier integrity. The untreated condition (“untreated”) refers to cells seeded in the transwell system without any treatment, it is used as reference for the TERR value. Error bars indicate standard deviation.

FIG. 10) shows effects of nanoparticles exposure on the translocation of lucifer yellow through the intestinal barrier. Differentiated Caco-2 (A-B) and differentiated Caco-2/HT29-MTX-E12 co-culture (C-D) were exposed to nanoparticles (NP-1 or NP-2), covering 100% of cell surface, for 24h (A-C) and 48h (B-D). The barrier integrity was assessed by the measurement of the translocation of lucifer yellow (LY) from the donor to acceptor compartment for 90 min. Histograms represent the percentage of the cumulative permeability of lucifer yellow (LY). EGTA (2.5 mM) is a calcium chelator that disrupts the tight junctions, it is used as control for the loss of barrier integrity. The untreated condition (“untreated”) refers to cells seeded in the transwell system without any treatment, it is used as reference for the diffusion of LY. Error bars indicate standard deviation.

FIG. 11) shows the evaluation of the cellular uptake of the nanoparticles in the intestinal barrier model. (A) Differentiated Caco-2/HT29-MTX-E12 co-culture were incubated with fluorescent-fully-chitosan-functionalized-shielded-nanoparticles (NP-2) for 24h. Cells were stained with phalloidin-TRITC (cellular membrane) and DAPI (nucleus). The localization of the nanoparticles in the intestinal barrier were assessed by confocal microscopy. The crossed line in the XY image marks the position of the XZ (below) and YZ (right) side views. (B) Differentiated Caco-2/HT29-MTX-E12 co-culture and THP-1 differentiated into M0 macrophages were treated with NP-2 for 24 h and analyzed by flow cytometry. Histograms represents the fluorescence of untreated- (dash line) and NP-2-treated-cells (full line). THP-1 are used as positive control for the internalization of the nanoparticles.

FIG. 12) shows the activity of immobilized and shielded pancreatin on fully-chitosan-functionalized-shielded-nanoparticles depending on the shield composition. The biocatalytic activity of fully-chitosan-functionalized-shielded-nanoparticles with immobilized and shielded pancreatin (NP-12, NP-14, and NP-16) was assessed for 24 h at 37° C. and compared to the free pancreatin. The activity of each enzyme comprised by pancreatin i.e., protease (A), lipase (B) and amylase (C), was assessed. Curves represents the percentage of remaining enzymatic activity compared to the initial activity. The nanoparticles used are: NP-14: Pancreatin-partially-shielded nanoparticles with a protection layer composed of APTES and TEOS, and functionalized with chitosan (A); NP-12: Pancreatin-fully-shielded-nanoparticles with a protection layer composed of APTES, TEOS, and Benzyltriethoxysilane, and functionalized with chitosan (B); NP-16: Pancreatin-fully-shielded-nanoparticles with a protection layer composed of APTES and TEOS, and functionalized with chitosan (C).

FIG. 13) shows the protective effect of chitosan on protease activity. The protease activity of immobilized and shielded pancreatin nanoparticles was assessed for 1 h at 37° C. in acidic conditions (acetic acid solution, pH:4) and compared to the activity of immobilized and shielded pancreatin nanoparticles in basic conditions (pH 8). Histograms represent the percentage of remaining activity compared to the initial activity. The nanoparticles used are: NP-13: Pancreatin-partially-shielded-nanoparticles with a protection layer composed of APTES and TEOS; NP-14: Pancreatin-partially-shielded-nanoparticles with a protection layer composed of APTES and TEOS, and functionalized with chitosan.

FIG. 14) shows ex-vivo interaction of thiol-functionalized-shielded-nanoparticles with mucus layer depending on the percentage of thiol-functionalization at the surface of nanoparticles. Fluorescent-thiol-functionalized-shielded-nanoparticles (500 μg/mL) were added to a layer of porcine mucus for 1 h. (A) Images show the nanoparticles (dark area within the circle) bound on the mucus layer after washing. The quantification of nanoparticles bound on the mucus layer is shown in the table (B and on the histograms (C). The different formulations of nanoparticles assessed are: NP-17: fluorescent-BSA-nanoparticles partially functionalized (5%) with MPTS; NP-18: fluorescent-BSA-nanoparticles partially functionalized (10%) with MPTS; NP-19: fluorescent-BSA-nanoparticles partially functionalized (20%) with MPTS, NP-20: fluorescent-BSA-nanoparticles partially functionalized (50%) with MPTS; NP-21: fluorescent-BSA-nanoparticles fully functionalized (100%) with MPTS.

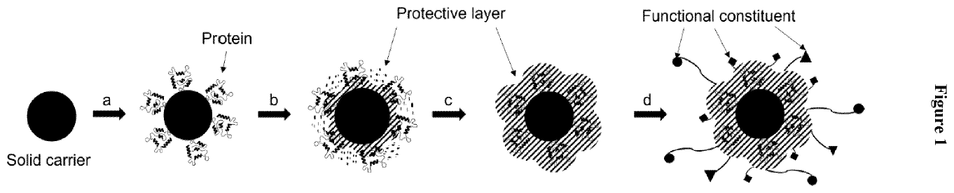

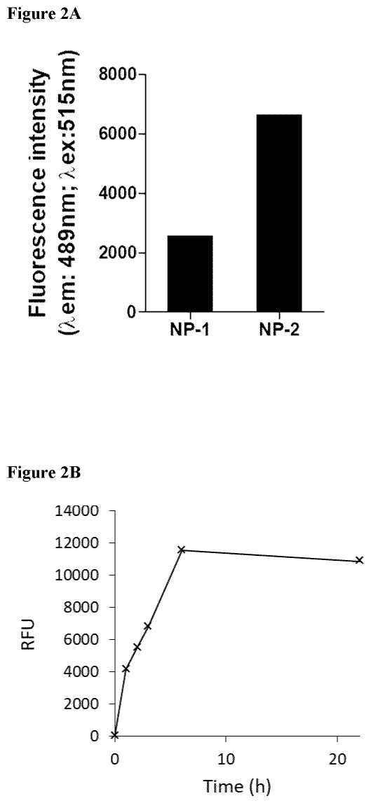

### DETAILED DESCRIPTION OF THE INVENTION

The present invention relates to a composition comprising a solid carrier, a protein or a fragment thereof immobilized on the surface of the solid carrier, a protective layer to protect the protein or a fragment thereof by embedding the protein or a fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

Features, integers, characteristics, compounds described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments.

The term “comprise” and variations thereof, such as, “comprises” and “comprising” is generally used in the sense of include, that is, as “including, but not limited to”, that is to say permitting the presence of one or more features or components.

The singular forms “a,” “an,” and “the” include plural referents unless the content clearly dictates otherwise.

The term “about” refers to a range of values±10% of a specified value. For example, the phrase “about 200” includes +10% of 200, or from 180 to 220.

The term “solid carrier” as used herein refers usually to a particle. Preferably the solid carrier is a monodisperse particle or a polydisperse particle, more preferably a monodisperse particle. The solid carrier usually comprises organic particles, inorganic particles, organic-inorganic particles, self-assembling organic particles, silica particles, gold particles, titanium particles and is preferably a silica particle, more preferably a silica nanoparticle (SNP). The particle size of the solid carrier is usually between and 1 nm and 1000 μm, preferably between 10 nm and 100 μm, particularly about 50 nm.

The term “linker” or “cross-linker” which are used synonymously herein refers to any linking reagents containing groups, which are capable of binding to specific functional groups (e.g. primary amines, sulfhydryls, etc.). A linker in the context of the present invention usually connects the surface of the solid carrier with the enzyme. For example, a linker may be immobilized on the surface of the solid carrier e.g. on the silica surface as a carrier material and then the enzyme may be bound to an unoccupied binding-site of the linker. Alternatively, the linker may firstly bind to the enzyme and then the linker bound to the enzyme may bind with its unoccupied binding-site to the solid carrier. Various types of linkers are known in the art, including but not limited to straight or branched-chain carbon linkers, heterocyclic carbon linkers, peptide linkers, polyether linkers, and linkers that are known in the art as tags.

The term “protective layer” as used herein refers to a layer for protecting the functional properties of the protein or fragment thereof e.g. the enzyme or fragment thereof immobilized on the surface of the solid carrier. The protective layer of the present invention is usually built with building blocks at least part of which are monomers capable of interacting with both each other usually by covalent binding and the immobilized enzyme usually by non-covalent binding. The protective layer is formed on the surface of the solid carrier to protect the protein or the fragment thereof immobilized on the solid carrier. The protective layers are usually homogeneous layers where at least 50%, preferably at least 70%, more preferably at least 90% of the protein or fragment thereof e.g. enzyme or fragment thereof are embedded in the protective layer.

The term “protein or fragment thereof” as used herein includes proteins comprising usually between 100 and 1500 amino acids, preferably between 100 and 800 amino acids, more preferably between 100 and 500 amino acids. A fragment of a protein as defined herein does usually have the same functional properties as the protein from which it is derived. A preferred protein or fragment thereof is an enzyme or a fragment thereof.

The term, “enzyme or a fragment thereof” includes naturally occurring enzymes or a fragment thereof and also includes artificially engineered enzymes or a fragment thereof. Artificially engineered enzymes or a fragment thereof are e.g. variants or functionally active fragments of the enzyme. By “variants or functionally active fragments thereof” in relation to the enzyme of the present invention is meant that the fragment or variant (such as an analogue, derivative or mutant) is capable of exercising the same physiological function as the enzyme. Such variants include naturally occurring allelic variants and non-naturally occurring variants. Additions, deletions, substitutions and derivatizations of one or more of the amino acids are contemplated so long as the modifications do not result in loss of functional activity of the fragment or variant. Preferably the functionally active fragment or variant has at least about 80% sequence identity more preferably at least about 90% sequence identity, even more preferably at least about 95% sequence identity, most preferably at least about 98% sequence identity to the relevant part of the enzyme. A fragment of an enzyme as defined herein does usually have the same functional properties as the enzyme from which it is derived.

The term “partially embedded protein” as used herein shall mean that the protein is not fully covered by the protective layer, thus, the protein is not fully embedded in the protective layer. In one embodiment less than 50% of the protein of interest are covered by the protective layer, though typically more at least 70% will be covered, thus improving protection of the protein. In a preferred embodiment, at least 70%, more preferably at least 80%, even more preferably at least 90%, most preferably at least 95% of the protein of interest is covered by the protective layer. In another preferred embodiment, around 70% to around 95%, more preferrably around 80% to around 95%, even more preferably around 90% to around 95%, most preferably around 90% to around 95, 96, 97, 98 or 99% of the protein of interest are covered by the protective layer. In a particularly preferred embodiment, around 70%, particularly around 80%, more particularly around 90%, most particularly around 95% of the protein of interest is covered by the protective layer. In a more particularly preferred embodiment, around 70%, particularly around 80%, more particularly around 90%, most particularly around 95% of the protein of interest is covered by the protective layer, wherein the active site is not covered.

The term “fully embedded protein” as used herein shall mean that the protein of interest according to the invention is fully, i.e. 100% covered by the protective layer, i.e. that also the active site is covered.

The term “at least partially embedded protein” as used herein shall mean that the protein is at least partially embedded and may be fully embedded by the protective layer. Thus “at least partially embedded protein” means that the protective layer covers from about 30% and 100% of the protein or a fragment thereof, preferably from about 50% to about 100%, more preferably from about 80% to about 100%, even more preferably from about 90% to about 100%, most preferably from about 95% to about 100%, wherein the active site is preferably covered.

The term “partially embedded enzyme” as used herein shall mean that the enzyme is not fully covered by the protective layer, thus, the enzyme is not fully embedded in the protective layer. In one embodiment less than 50% of the enzyme of interest are covered by the protective layer, though typically more at least 70% will be covered, thus improving protection of the enzyme. In a preferred embodiment, at least 70%, more preferably at least 80%, even more preferably at least 90%, most preferably at least 95% of the enzyme of interest is covered by the protective layer. In another preferred embodiment, around 70% to around 95%, more preferrably around 80% to around 95%, even more preferably around 90% to around 95%, most preferably around 90% to around 95, 96, 97, 98 or 99% of the enzyme of interest are covered by the protective layer. In a particularly preferred embodiment, around 70%, particularly around 80%, more particularly around 90%, most particularly around 95% of the enzyme of interest is covered by the protective layer. In a more particularly preferred embodiment, around 70%, particularly around 80%, more particularly around 90%, most particularly around 95% of the enzyme of interest is covered by the protective layer, wherein the active site is not covered.

The term “fully embedded enzyme” as used herein shall mean that the enzyme of interest according to the invention is fully, i.e. 100% covered by the protective layer, i.e. that also the active site is covered.

The term “at least partially embedded enzyme” as used herein shall mean that the enzyme is at least partially embedded and may be fully embedded by the protective layer. Thus “at least partially embedded enzyme” means that the protective layer covers from about 30% and 100% of the enzyme or a fragment thereof, preferably from about 50% to about 100%, more preferably from about 80% to about 100%, even more preferably from about 90% to about 100%, most preferably from about 95% to about 100%, wherein the active site is preferably covered.

The term “functional constitutent” as used herein refers to a constituent which after being immobilized to the surface of the protective layer retains its characteristic, functional property. A functional constituent in the sense of the present invention is a polymer comprising repeat units wherein each repeat unit comprises at least one amino group and/or at least one thiol group.

The term “polymer comprising repeat units wherein each repeat unit comprises at least one amino group” as used herein refers to a polymer comprising a number of repeat units (monomers), wherein each repeat unit comprises at least one amino group. A preferred polymer comprises a number of repeat units (monomers), wherein each repeat unit contains one amino group, in particular one primary amino group.

The term “polymer comprising repeat units wherein each repeat unit comprises at least one thiol group” as used herein refers to a polymer comprising a number of repeat units (monomers), wherein each repeat unit comprises at least one thiol. A preferred polymer comprises a number of repeat units (monomers), wherein each repeat unit contains one thiol group.

The term “polycarbophil-cysteine conjugates” as used herein refers to conjugates which comprise cysteine covalently attached to polycarbophil. Such conjugates can be produced as referred in e.g. Bernkop-Schnurch and Thaler, 2000, Journal of Pharmaceutical Sciences 89(7):901-9.

The term “polylysine” as used herein refers to a-polylysine and or ε-polylysine (ε-poly-L-lysine, EPL), preferably ε-polylysine. α-polylysine is a synthetic polymer, which can be composed of either L-lysine or D-lysine. ε-polylysine (ε-poly-L-lysine, EPL) is typically produced as a homopolypeptide of approximately 25-30 L-lysine residues.

The term “polycysteine” as used herein can be composed of either L-cysteine or D-cysteine and is preferably composed of L-cysteine and comprises preferably between 2 and 30 cysteine residues, more preferably between 2 and 5 cysteine residues.

The term “polyglucosamin” as used herein refers to linear amino-polysaccharides composed of D-glucosamine and N-acetyl-D-glucosamine units linked by (1-4) glycosidic bonds. Polyglucosamine contains free amine (—NH2) groups and may be characterized by the proportion of N-acetyl-D-glucosamine units and D-glucosamine units, which is expressed as the degree of deacetylation (DDA) of the fully acetylated polymer chitin. A preferred polyglucosamin of the present invention is selected from the group consisting of chitin, chitosan, polyglucosaminoglycans, chondroitin, heparin, keratan and dermatan or a derivative thereof. Most preferred is a chitosan or a derivative thereof.

The term “chitosan or a derivative thereof” as used herein refers to a chitosan or chitosan derivative thereof including a salt thereof which has preferably a molecular weight of 2 000 Da or more, preferably in the range 25 000-2 000 000 Da and more preferably about 50 000-350 000 Da, most preferably about 50 000-190 000 Da or 190 000-310 000 Da. The term chitosan derivatives includes ester, ether or other derivatives formed by reaction of acyl or alkyl groups with the OH groups. Examples are O-alkyl ethers of chitosan, O-acyl esters of chitosan. Suitable derivatives are given e.g. in G. A. E. Roberts, Chitin Chemistry, MacMillan Press Ltd, London, 1992. Suitable salts of chitosan include nitrates, phosphates, sulphates, xanthates, hydrochlorides, glutamates, lactates, acetates.

In a first aspect the present invention provides a composition comprising a solid carrier, a protein or a fragment thereof immobilized on the surface of the solid carrier, a protective layer to protect the protein or a fragment thereof by embedding the protein or a fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group.

The protein or fragment thereof, e.g. the enzyme or fragment thereof can be immobilized on the surface of the solid carrier by non-covalent binding or covalent binding. Non-covalent binding includes p-p (aromatic) interactions, van der Waals interactions, H-bonding interactions, and electrostatic interactions like e.g. ionic interactions. Preferably, the protein or fragment thereof, e.g. the enzyme or fragment thereof is immobilized on the surface of the solid carrier by covalent binding or by covalent binding via a linker.

In one embodiment the solid carrier is selected from the group of organic particles, inorganic particles, organic-inorganic particles, self-assembling organic particles, silica particles, gold particles, titanium particles and is preferably a silica particle, more preferably a silica nanoparticle (SNP). The particle size is usually measured by measuring the diameter of the particles and is usually between 1 nm and 1000 nm, preferably between 10 nm and 100 nm, particularly about 50 nm. In case the solid carrier is a monodisperse particle, the size is usually between 1 nm and 1000 nm, preferably between 10 nm and 100 nm, particularly about 50 nm. In case the solid carrier is a polydisperse particle, the size is usually between 1 nm and 1000 μm, preferably between 10 nm and 100 μm, particularly between 50 nm and 50 μm.

Usually monodisperse particles or polydisperse particles, preferably monodisperse particles are used as solid carrier in the present invention. In a preferred embodiment the monodisperse particles are spherical monodisperse particles. In a further preferred embodiment, the polydisperse particles are non-spherical polydisperse particles.

The solid carrier is usually provided in suspension. Suspension of the solid carrier can be e.g. in water, buffer or non-ionic surfactants or mixtures thereof, preferably in mixtures of water and non-ionic surfactants. Buffers which can be used in the method of the present invention are phosphate, piperazine-N,N′-bis(2-ethanesulfonic acid), 2-Hydroxy-3-morpholinopropanesulfonic acid, N,N-bis[2-hydroxyethyl]-2-aminoethanesulfonic acid), (3-(N-morpholino)propanesulfonic acid), 2-[[1,3-dihydroxy-2-(hydroxymethyl)propan-2-yl]amino]ethanesulfonic acid, 4-(2-hydroxyethyl)-1-piperazineethanesulfonic acid), 3-(N,N-Bis[2-hydroxyethyl]amino)-2-hydroxypropanesulfonic acid, N,N-Bis(2-hydroxyethyl)-3-amino-2-hydroxypropanesulfonic acid, N-[Tris(hydroxymethyl)methyl]glycine, Diglycine, 4-(2-Hydroxyethyl)-1-piperazinepropanesulfonic acid, N,N-Bis(2-hydroxyethyl)glycine, N-[Tris(hydroxymethyl)methyl]-3-aminopropanesulfonic acid, N-(1,1-Dimethyl-2-hydroxyethyl)-3-amino-2-hydroxypropanesulfonic acid.

In one embodiment the surface of the solid carrier is modified to introduce a molecule or functional chemical group as anchoring point i.e. as anchoring point for the protein e.g. the enzyme or for the linker connecting the protein e.g. the enzyme to the solid carrier. Preferably, said anchoring point is an amine functional chemical group or moiety. As a non-limiting example, an amino-modified surface of the solid carrier e.g. an amino-modified silica surface may be used as modified solid carrier. Such an amino-modified surface of the solid carrier may be obtained by reacting a solid carrier having a silica surface with an amino silane, e.g. with APTES. Thus in a preferred embodiment, the solid carrier is a solid carrier having a silica surface with an amino-modified surface, more preferably a solid carrier obtained by reacting the solid carrier having a silica surface with an amino silane, e.g. with APTES. Such a modified carrier may form an amide linkage between the protein e.g. the enzyme and the amine group at the surface of the carrier material or an amide linkage between the linker and the amine group at the surface of the carrier material. In one embodiment the introduced molecule or functional chemical group as anchoring point is homogeneously distributed on the surface of the solid carrier.

In some embodiments the protective layer has a defined thickness of about 1 to about 200 nm, usually 1 to about 100 nm, preferably about 1 to about 50 nm, more preferably about 1 to about 25 nm, even more preferably about 1 to about 20 nm, in particular about 1 to about 15 nm. The most preferred defined thickness is about 1 to about 10 nm. In some embodiments the layer has a defined thickness of about 5 to about 100 nm, preferably about 5 to about 50 nm, more preferably about 5 to about 25 nm, even more preferably about 5 to about 20 nm, in particular about 5 to about 15 nm. The most preferred defined thickness is about 5 to about 10 nm. The protective layer is usually porous and the pore size is between 1 and 100 nm, preferably between 1 and 20 nm.

In one embodiment, the protein or fragment thereof, e.g. the enzyme or a fragment thereof is partially embedded by the protective layer. In a preferred embodiment the protein or fragment thereof, e.g. the enzyme or a fragment thereof is at least partially embedded by the protective layer. In a more preferred embodiment the protein or fragment thereof, e.g. the enzyme or a fragment thereof is fully embedded by the protective layer.

In one embodiment, the protective layer embeds the solid carrier and embeds the protein or fragment thereof, e.g. the enzyme or a fragment thereof immobilized on the surface of the solid carrier. In one embodiment, the functional constituent immobilized on the surface of the protective layer, is not embedded by the protective layer. Preferably, the protective layer fully embeds the solid carrier and fully embeds the protein or fragment thereof, e.g. the enzyme or a fragment thereof immobilized on the surface of the solid carrier. More preferably, the protective layer fully embeds the solid carrier and fully embeds the protein or fragment thereof, e.g. the enzyme or a fragment thereof immobilized on the surface of the solid carrier and the functional constituent immobilized on the surface of the protective layer is not embedded by the protective layer. If the protective layer fully embeds the solid carrier and fully embeds the protein or fragment thereof, e.g. the enzyme or a fragment thereof immobilized on the surface of the solid carrier, the protein or fragment thereof, e.g. the enzyme or fragment thereof is fully, i.e. 100% covered by the protective layer, i.e. that also the active site is covered and the solid carrier is fully, i.e. 100% covered by the protective layer.

In a preferred embodiment the protein or a fragment thereof is an enzyme or a fragment thereof.

In a more preferred embodiment the protein or a fragment thereof is selected from the group consisting of serum albumin or a fragment thereof, lipase or a fragment thereof, pancreatin and a protein or fragment thereof comprised by pancreatin. A protein or fragment thereof comprised by pancreatin is usually a protein or fragment thereof selected from the group consisting of proteases, amylases and lipases.

In an even more preferred embodiment the protein or a fragment thereof is selected from the group consisting of serum albumin or a fragment thereof, lipase or a fragment thereof, and pancreatin.

In a particular preferred embodiment the protein or a fragment thereof is lipase or a fragment thereof.

In one embodiment the enzyme or a fragment thereof is selected from the group consisting of oxidoreductases, transferases, hydrolases, lyases, isomerases, transpeptidases, or ligases, or a fragment thereof and mixtures thereof. Particular preferred is a hydrolase or a fragment thereof, more particular a lipase or a fragment thereof.

The protective layer thickness can be measured, by using a microscope such as scanning electron microscope (SEM), transmission electron microscopy (TEM), scanning probe microscopy (SPM), light scattering methods or by ellipsometry.

The composition of the present invention is usually produced in a reaction vessel like a reactor. The formation of the protective layer is usually carried out by forming the respective protective layer by building blocks, wherein the building blocks build the protective layer in a polycondensation reaction. The polycondensation can be effected in different solvents, preferably in aqueous solution. Polycondensation can be easily controlled and stopped if appropriate, allowing achievement of a defined thickness of the protective layer. The choice of the building blocks, which can be used to build the protective layer, may depend on the known structure of the protein e.g. the enzyme in order to adapt the affinity of the protective layer according to optimal and/or desired parameters. As building blocks for the protective layer usually structural building blocks and protective building blocks are used to build the protective layer. Structural building blocks which can be used are e.g. tetraethylorthosilicate (designated herein as “TEOS” or “T”). Protective building blocks which can be used are e.g. 3-Aminopropyltriethoxysilane (designated herein as “APTES” or “A”), Propyltriethyoxysilane (designated herein as “PTES” or P”), Isobutyltriethoxysilane (designated as “IBTES”), Hydroxymethyltriethoxysilane (designated herein as “HTMEOS” or H), Benzyltriethoxysilane (designated herein as “BTES”), Ureidopropyltriethoxysilane (designated as “UPTES”), or Carboxyethyltriethoxysilane (designated herein as “CETES”). Structural building blocks are usually precursors of inorganic silica, capable of forming 4 covalent bonds in the layer formed. Protective building blocks are usually organosilanes, bearing an organic moiety endowed with the ability to interact with the proteins e.g. the enzymes (e.g., enzyme). Preferred structural building blocks are tetravalent silanes, in particular tetra-alkoxy-silanes. Preferred protective building blocks are trivalent silanes, in particular tri-alkoxy-silanes. More preferred structural building blocks are mixtures of tetravalent silanes and trivalent silanes, in particular mixtures of tetra-alkoxy-silanes and tri-alkoxy-silanes. Even more preferred structural building blocks are selected from the group consisting of tetraethylorthosilicate, tetra-(2-hydroxyethyl)silane, and tetramethylorthosilicate. Even more preferred protective building blocks are selected from the group consisting of carboxyethylsilanetriol, benzylsilanes, propylsilanes, isobutylsilanes, n-octylsilanes, hydroxysilanes, bis(2-hydroxyethyl)-3-aminopropylsilanes, aminopropylsilanes, ureidopropylsilanes, (N-Acetylglycyl)-3-aminopropylsilanes, hydroxy(polyethyleneoxy)propyl]triethoxysilanes, in particular selected from benzyltriethoxysilane, propyltriethoxysilane, isobutyltriethoxysilane, n-octyltriethoxysilane, hydroxymethyltriethoxysilane, bis(2-hydroxyethyl)-3-aminopropyltriethoxysilane, 3-Aminopropyltriethoxysilane, ureidopropyltriethoxysllane, (N-Acetylglycyl)-3-aminopropyltriethoxysilane, or selected from benzyltrimethoxysflane, propyltrimethoxysilane, isobutylimethoxysilane, n-octyltrimethoxysilane, hydroxyrnethyltrimethoxysilane, bis(2-hydroxyethyl)-3-aminopropyltrimethoxysilane, arninopropyltrimethoxysilane, ureidopropyltrimethoxysilane (N-Acetylglycyl)-3-aminopropyltrimethoxysilane or selected from benzyltrihydroxyethoxysilane, propyltrihydroxyethoxysilane, isobutyltrihydroxyethoxysilane, n-octyltrihydroxyethoxysilane, hydroxymefilyltrihydroxyethoxysilane, bis(2-hydroxyethyl)-3-aminopropyltrihydroxyethoxysilane, aminopropyltrihydroxyethoxysilane, Ureidopropyltrihydroxyethoxysilane (N-Acetylglycyl)-3-aminopropyltrihydroxymethoxysilane.

Particular preferred building blocks are TEOS as structural building block and APTES, PTES, and/or HTMEOS, preferably APTES as protective building block. In particular TEOS as structural building block and APTES as protective building block are used to build the protective layer.

The reaction time of the building blocks with the solid carrier depends on the length of the linker, if a linker is used, and the size of the protein e.g. the enzyme. The reaction is usually carried out for a time period of between 0.5 to 10 hours, preferably between 1 and 5 hours, more preferably between 1 and 4 hours, even more preferably between 2 and 4 hours, preferably in aqueous solution and preferably at room temperature of about 5 to about 25° C. or at about 20° C. The formation of the protective layer can be stopped by actively stopping the polycondensation reaction e.g by removing the non-reacted building blocks e.g. by a washing step or by self-stopping of the polycondensation reaction caused by a limited amount of building blocks.

In a further more preferred embodiment the protein e.g. the enzyme is immobilized on the solid carrier by at least partly modifying the surface of the solid carrier by introducing a molecule as anchoring point as described supra for the protein e.g. the enzyme and by using a linker, preferably a cross-linker binding to the anchoring point and the protein e.g. the enzyme.

In one embodiment the introduced molecule as anchoring point and/or the linker are homogeneously distributed on the surface of the solid carrier.

In a preferred embodiment the cross-linker is selected from the group consisting of glutaraldehyde, disuccinimidyl tartrate, bis[sulfosuccinimidyl]suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulfhydrils, sulfhydryl-reactive 2-pyridyldithiol, BSOCOES (Bis[2-(succinimidooxycarbonyloxy)ethyl]sulfone), DSP (Dithiobis[succinimidyl]propionate]), DTSSP (3,3′-Dithiobis[sulfosuccinimidyl]propionate]), DTBP (Dimethyl 3,3′-dithiobispropionimidate 2 HCl), DST (Disuccinimidyl tartarate), Sulfo-LC-SMPT (4-Sulfosuccinimidyl-6-methyl-a-(2-pyridyldithio)toluamido]hexanoate)), SPDP (N-Succinimidyl 3-(2-pyridyldithio)-propionate), LC-SPDP (Succinimidyl 6-(3-[2-pyridyldithio]-propionamido)hexanoate), SMPT (4-Succinimidyloxycarbonyl-methyl-a-[2-pyridyldithio]toluene), DPDPB (1,4-Di-[3′-(2′-pyridyldithio)-propionamido]butane), DTME (Dithio-bismaleimidoethane), BMDB (1,4 bismaleimidyl-2,3-dihydroxybutane). More preferably said cross-linker is selected from glutaraldehyde, disuccinimidyl tartrate, disuccinimidyl suberate, bis[sulfosuccinimidyl]suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulfhydrils (e.g. sulfhydryl-reactive 2-pyridyldithio). Most preferred is glutaraldehyde.

After the protective layer has been formed, the solid carrier comprising the protein e.g. the enzyme and the protective layer can be stored. Storing is usually accomplished e.g. by washing the composition formed e.g. with a buffer and storing it suspended or solved in that buffer for a desired time period. In a preferred embodiment the solid carrier comprising the protein e.g. the enzyme and the protective layer is stored at a constant temperature between 2 to 25° C. In a further preferred embodiment, the solid carrier comprising the enzyme and the protective layer is stored 5 to 48 hours, preferably 10 to 30 hours. More preferably the solid carrier comprising the protein e.g. the enzyme and the protective layer is stored at a constant temperature between 2 to 25° C., preferably at room temperature for 10 to 30 hours.

In one embodiment, the functional constituent binds to mucus.

In one embodiment a polymer comprising repeat units wherein each repeat unit comprises at least one amino group and/or at least one thiol group is a polymer comprising repeat units wherein each repeat unit comprises at least one amino group.

In one embodiment a polymer comprising repeat units wherein each repeat unit comprises at least one amino group and/or at least one thiol group is a polymer comprising repeat units wherein each repeat unit comprises at least one thiol group.

In one embodiment a polymer comprising repeat units wherein each repeat unit comprises at least one amino group and/or at least one thiol group is selected from the group consisting of a polyglucosamin, a polymerized silane-PEG-NH2 and a polymerized silane comprising an amino group. In a preferred embodiment the polymer comprising repeat units wherein each repeat unit comprises at least one amino group and/or at least one thiol group, is selected from the group consisting of a polyglucosamin, a polymerized silane-PEG-NH2 and polymerized APTES.

In a more preferred embodiment the polymer comprising repeat units wherein each repeat unit comprises at least one amino group and/or at least one thiol group is selected from the group consisting of a polyglucosamin selected from the group consisting of chitin, chitosan, polyglucosaminoglycans, chondroitin, heparin, keratan and dermatan or a derivative thereof, a polymerized silane-PEG-NH2; and a polymerized silane comprising an amino group, preferably a polymerized APTES. In an even more preferred embodiment the polymer comprising repeat units wherein each repeat unit comprises at least one amino group and/or at least one thiol group, is a polyglucosamin, preferably a polyglucosamin selected from the group consisting of chitin, chitosan, polyglucosaminoglycans, chondroitin, heparin, keratan and dermatan or a derivative thereof, more preferably a chitosan or a derivative thereof.

A preferred polyglucosamin of the present invention is selected from the group consisting of chitin, chitosan, polyglucosaminoglycans, chondroitin, heparin, keratan and dermatan or a derivative thereof. Most preferred is a chitosan or a derivative thereof. A preferred silane-PEG-NH2 of the polymerized silane-PEG-NH2 is selected from the group consisting of silane-PEG4-NH2, silane-PEG2000-NH2, and silane-PEG5000-NH2. A preferred polymerized silane comprising an amino group is selected from the group consisting of APTES, amino-butyl-TES, amino-pentyl-TES, amino-hexyl-TES, amino-heptyl-TES, and amino-octyl-TES, and is in particular APTES.

In a further embodiment a polymer comprising repeat units wherein each repeat unit comprises at least one amino group and/or at least one thiol group is selected from the group consisting of a polyglucosamin, a polymerized silane-PEG-NH2, a polymerized silane comprising an amino group, a polymerized silane comprising a thiol group, a polycarbophil-cysteine conjugate, a polymerized silane-PEG-thiol and a polycysteine. In a further more preferred embodiment the polymer comprising repeat units wherein each repeat unit comprises at least one amino group and/or at least one thiol group is selected from the group consisting of a polyglucosamin selected from the group consisting of chitin, chitosan, polyglucosaminoglycans, chondroitin, heparin, keratan and dermatan or a derivative thereof, a polymerized silane-PEG-NH2; a polymerized silane comprising a thiol group, preferably a polymerized MPTS; a polycarbophil-cysteine conjugate; a polymerized silane-PEG-thiol; and a polycysteine. In an even more preferred embodiment the polymer comprising repeat units wherein each repeat unit comprises at least one amino group and/or at least one thiol group, is a polyglucosamin or a polymerized silane comprising a thiol group, preferably a polyglucosamin selected from the group consisting of chitin, chitosan, polyglucosaminoglycans, chondroitin, heparin, keratan and dermatan or a derivative thereof, more preferably a chitosan or a derivative thereof or a polymerized silane comprising a thiol group, a polycarbophil-cysteine conjugate, and a polymerized silane-PEG-thiol, preferably a polymerized silane comprising a thiol group.

In a particular embodiment, the polymer comprising repeat units wherein each repeat unit comprises at least one amino group and/or at least one thiol group, is selected from the group consisting of chitin, chitosan, polyglucosaminoglycans, chondroitin, heparin, keratin, dermatan or a derivative thereof in particular chitosan or a derivative thereof, a polymerized silane-PEG-NH2 selected from the group consisting of polymerized silane-PEG4-NH2, polymerized silane-PEG2000-NH2, polymerized silane-PEG5000-NH2, a polymerized silane comprising an amino group which is preferably polymerized APTES and a polymerized silane comprising a thiol group, which is preferably polymerized MPTS.

In one embodiment a polymer comprising repeat units wherein each repeat unit comprises at least one amino group and/or at least one thiol group is selected from the group consisting of a polyglucosamin, a polymerized silane-PEG-NH2, a polymerized silane comprising an amino group and a polymerized silane comprising a thiol group. In a preferred embodiment the polymer comprising repeat units wherein each repeat unit comprises at least one amino group and/or at least one thiol group, is selected from the group consisting of a polyglucosamin, a polymerized silane-PEG-NH2, polymerized APTES and polymerized MPTS. In a more preferred embodiment the polymer comprising repeat units wherein each repeat unit comprises at least one amino group and/or at least one thiol group is selected from the group consisting of a polyglucosamin selected from the group consisting of chitin, chitosan, polyglucosaminoglycans, chondroitin, heparin, keratan and dermatan or a derivative thereof; a polymerized silane-PEG-NH2; a polymerized silane comprising an amino group, preferably a polymerized APTES; and a polymerized silane comprising a thiol group, preferably polymerized MPTS. In a particular embodiment, the polymer comprising repeat units wherein each repeat unit comprises at least one amino group and/or at least one thiol group, is selected from the group consisting of chitin, chitosan, polyglucosaminoglycans, chondroitin, heparin, keratin, dermatan or a derivative thereof, in particular chitosan or a derivative thereof, a polymerized silane-PEG-NH2 selected from the group consisting of polymerized silane-PEG4-NH2, polymerized silane-PEG2000-NH2, polymerized silane-PEG5000-NH2, a polymerized silane comprising an amino group which is APTES and a polymerized silane comprising an thiol group which is MPTS.

In one embodiment a polymer comprising repeat units wherein each repeat unit comprises at least one thiol group is selected from the group consisting of a polymerized silane comprising a thiol group, a polycarbophil-cysteine conjugate, a polymerized silane-PEG-thiol and a polycysteine, and is preferably selected from the group consisting of a polymerized silane comprising a thiol group, a polycarbophil-cysteine conjugate, and a polymerized silane-PEG-thiol, and is more preferably a polymerized silane comprising a thiol group, and is most preferably polymerized MPTS. In one embodiment a polymerized silane comprising a thiol group is preferably polymerized MPTS.

In one embodiment 5% to 100%, preferably 10% to 100%, more preferably 50% to 100%, of the surface of the protective layer is covered with a polymer comprising repeat units wherein each repeat unit comprises at least one amino group and/or at least one thiol group.

In one embodiment the functional constituent is immobilized on the surface of the protective layer by binding, preferably covalent binding. In a preferred embodiment the functional constituent is immobilized on the surface of the protective layer by non-covalent binding, preferably by electrostatic interactions. In a more preferred embodiment the polymer comprising repeat units wherein each repeat unit comprises at least one amino group and/or at least one thiol group is immobilized on the surface of the protective layer by covalent binding.

In one embodiment the functional constituent is immobilized on the surface of the protective layer using a spacer binding to the surface of the protective layer and the functional constituent. Thus in one embodiment the present invention comprises a composition comprising a solid carrier, a protein or a fragment thereof immobilized on the surface of the solid carrier, a protective layer to protect the protein or a fragment thereof by embedding the protein or a fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group, wherein the functional constituent is immobilized on the surface of the protective layer by a spacer. Examples of such a spacer include a polyethylene such as PEG4, PEG2000, PEG5000. A functional constituent immobilized on the surface of the protective layer, by a spacer is usually produced by firstly reacting the spacer with the functional constituent, so that the spacer binds to the functional constituent and then the functional constituent bound to the spacer is reacted with the surface of the protective layer.

The immobilization of the functional constituent to the surface of the protective layer is usually carried out in a reaction vessel like a reactor by suspending the solid carrier carrying the protein e.g. the enzyme embedded in a protective layer as described supra in e.g. in water, buffer or non-ionic surfactants or mixtures thereof, preferably in mixtures of water and non-ionic surfactants. The functional component is then added to the suspension to react usually under stirring with the surface of the protective layer to immobilize the functional constitutent on the surface of the protective layer. Usually such obtained composition is washed and resuspended into water, buffer or non-ionic surfactants or mixtures thereof. Immobilization takes place by non-covalent binding e.g. electrostatic binding or by covalent binding of the functional constituent. t. The functional constituent may be immobilized by chemically modifying the surface of the protective layer and the functional constituent using e.g. “click chemistry” such as copper-catalyzed click chemistry (Copper-catalysed azide-alkyne cycloaddition, see e.g. Kolb et al. (2001) Angew. Chem. 40(11)2004-2021) or by copper free click chemistry (Wittig G, A Chem Ber, 1961, 94, 3260)., e.g. the solid carrier carrying the protein e.g. the enzyme embedded in a protective layer as described supra is first reacted with a reactive compound like an ethynyl compound and the functional constituent is modified by adding a reactive compound e.g. an azide residue and then both components are reacted to immobilize the functional constituent on the surface of the protective layer.

In a further embodiment the composition further comprises a chelating agent, wherein the chelating agent optionally comprises a radioactive or luminescent label. Preferably a chelating agent is selected from the group consisting of DOTA, DTPA, NOTA, TETA, AAZTA, TRAP, NOPO and HEHA. More preferably DOTA or HEHA are used. Even more preferably a chelating agent which comprises a radioactive or luminescent label is used, in particular p-SCN-Bn-DOTA or Lutetium-177-radiolabeled-DOTA is used. If the composition further comprises a chelating agent, the solid carrier carrying the protein e.g. the enzyme embedded in a protective layer is usually pretreated with a chelating agent and a different chelating agent which comprises a radioactive or luminescent label is added to the such pretreated composition.

Preferably a radioactive label is used, more preferably a compound of the lanthanides family, even more preferably Gadolinium, Lutetium, or Europium.

In a further aspect the present invention provides the composition as described supra for use as a medicament.

In a further aspect the present invention provides the composition for use in a method of enzyme replacement therapy (ERT), preferably gastrointestinal enzyme replacement therapy, or for use in a method for the prevention, delay of progression or treatment of exocrine pancreatic insufficiency (EPI), lactase deficiency, sucrase-isomaltase deficiency, disaccharidoses intolerances, peptides allergies, inflammatory bowel disease (IBD), cystic fibrosis, and/or a lung disease or disorder selected from the group consisting of Gaucher, Fabry and mucopolysaccharidosis (MPS).

Also provided is the use of the composition as described herein for the manufacture of a medicament for the prevention, delay of progression or treatment of exocrine pancreatic insufficiency (EPI), lactase deficiency, sucrase-isomaltase deficiency, disaccharidoses intolerances, peptides allergies, inflammatory bowel disease (IBD), cystic fibrosis, and/or a lung disease or disorder selected from the group consisting of Gaucher, Fabry and mucopolysaccharidosis (MPS). in a subject. Also provided is the use of the composition as described herein for the prevention, delay of progression or treatment of exocrine pancreatic insufficiency (EPI), lactase deficiency, sucrase-isomaltase deficiency, disaccharidoses intolerances, peptides allergies, inflammatory bowel disease (IBD), cystic fibrosis, and/or a lung disease or disorder selected from the group consisting of Gaucher, Fabry and mucopolysaccharidosis (MPS) in a subject. Also provided is a method for the prevention, delay of progression or treatment of exocrine pancreatic insufficiency (EPI), lactase deficiency, sucrase-isomaltase deficiency, disaccharidoses intolerances, peptides allergies, inflammatory bowel disease (IBD), cystic fibrosis, and/or a lung disease or disorder selected from the group consisting of Gaucher, Fabry and mucopolysaccharidosis (MPS) in a subject, comprising administering to said subject a therapeutically effective amount of the composition as described herein. Also provided herein is the use of the composition as described herein for the manufacture of a medicament for a method of enzyme replacement therapy (ERT), preferably gastrointestinal enzyme replacement therapy. Also provided is the use of the composition as described herein in a method of enzyme replacement therapy (ERT), preferably gastrointestinal enzyme replacement therapy in a subject. Also provided is a method of enzyme replacement therapy (ERT), preferably gastrointestinal enzyme replacement therapy, in a subject, comprising administering to said subject a therapeutically effective amount of the composition as described herein.

The expression “effective amount” or “therapeutically effective amount” as used herein refers to an amount capable of invoking one or more of the desired effects in a subject receiving the composition of the present invention. Determination of a therapeutically effective amount is well within the capability of those skilled in the art, especially in light of the detailed disclosure provided herein.

In a preferred embodiment the present invention provides the composition for use in a method for the prevention, delay of progression or treatment of exocrine pancreatic insufficiency (EPI), lactase deficiency, sucrase-isomaltase deficiency, disaccharidoses intolerances, peptides allergies, inflammatory bowel disease (IBD), and cystic fibrosis, more preferably for use in a method for the prevention, delay of progression or treatment of exocrine pancreatic insufficiency (EPI), lactase deficiency, sucrase-isomaltase deficiency, disaccharidoses intolerances, inflammatory bowel disease (IBD), and cystic fibrosis.

In a further preferred embodiment the present invention provides the composition for use in a method of enzyme replacement therapy (ERT), preferably gastrointestinal enzyme replacement therapy.

In a further preferred embodiment the present invention provides the composition for use in a method for the prevention, delay of progression or treatment of a lung disease or disorder selected from the group consisting of Gaucher, Fabry and mucopolysaccharidosis (MIPS).

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

As used herein, “delay of progression” means increasing the time to appearance of a symptom of e.g. a lung disease or disorder or cystic fibrosis ior a mark associated with e.g. a lung disease or disorder or cystic fibrosis or slowing the increase in severity of a symptom of e.g. a lung disease or disorder or cystic fibrosis. Further, “delay of progression” as used herein includes reversing or inhibition of disease progression. “Inhibition” of disease progression or disease complication in a subject means preventing or reducing the disease progression and/or disease complication in the subject.

Preventive treatments comprise prophylactic treatments. In preventive applications, the pharmaceutical combination of the invention is administered to a subject suspected of having, or at risk for developing the above mentioned diseases or disorders e.g. lung disease or disorder or cystic fibrosis. In therapeutic applications, the pharmaceutical combination is administered to a subject such as a patient already suffering from the above mentioned diseases or disorders e.g. lung disease or disorder or cystic fibrosis, in an amount sufficient to cure or at least partially arrest the symptoms of the disease. Amounts effective for this use will depend on the severity and course of the disease, previous therapy, the subject's health status and response to the drugs, and the judgment of the treating physician.

In the case wherein the subject's condition does not improve, the pharmaceutical combination of the invention may be administered chronically, which is, for an extended period of time, including throughout the duration of the subject's life in order to ameliorate or otherwise control or limit the symptoms of the subject's disease or condition.

In the case wherein the subject's status does improve, the pharmaceutical combination may be administered continuously; alternatively, the dose of drugs being administered may be temporarily reduced or temporarily suspended for a certain length of time (i.e., a “drug holiday”). Once improvement of the patient's condition has occurred, a maintenance dose of the pharmaceutical combination of the invention is administered if necessary. Subsequently, the dosage or the frequency of administration, or both, is optionally reduced, as a function of the symptoms, to a level at which the improved disease is retained.

In a further aspect the present invention provides a method of producing a composition as described supra, e.g. a composition comprising a solid carrier, a protein or a fragment thereof immobilized on the surface of the solid carrier, a protective layer to protect the protein or a fragment thereof by embedding the protein or a fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group; the method comprising the following steps:
(a) providing a solid carrier;
(b) immobilizing a protein or a fragment thereof on the solid carrier;
(c) forming a protective layer on the surface of the solid carrier to protect the protein or the fragment thereof immobilized on the solid carrier;
(d) immobilizing a functional constituent on the surface of the protective layer, wherein the functional constituent immobilized on the surface of the protective layer is a polymer comprising repeat units wherein each repeat unit comprises at least one amino group and/or at least one thiol group.

Step (a) is usually carried out by providing the solid carrier in suspension in water or a buffer. The suspension can be stirred e.g at 400 rpm, 20° C. for 30 min. The immobilization of the protein e.g. the enzyme on the solid carrier in step b) of the present method is usually carried out by adding a solution of the protein e.g. the enzyme to the suspension of the solid carrier. In a preferred embodiment the immobilization of the protein e.g. the enzyme on the solid carrier is carried out by providing a suspension of the solid carrier and adding a solution of the protein e.g. the enzyme, wherein the suspension with the added solution of the protein e.g. the enzyme is incubated to allow the enzyme to bind on the surface of the solid carrier. In a preferred embodiment the surface of the solid carrier is at least partly modified to improve immobilization of the protein e.g. the enzyme on the solid carrier. In particular, the surface of the solid carrier is at least partly modified before the protein e.g. the enzyme is immobilized. The surface of the solid carrier can be at least partly modified by introducing a molecule as anchoring point for the protein e.g. the enzyme to the surface of the solid carrier as described supra. The formation of the protective layer according to step (c) of the present method is usually carried out by forming the respective protective layer with building blocks, wherein the building blocks build the protective layer in a polycondensation reaction as described supa. The immobilization of a functional constituent on the surface of the protective layer according to step (d) of the present method is usually carried out as described supra.

In one embodiment the protective layer is formed by building blocks, wherein as building blocks structural building blocks and protective building blocks are used to form the protective layer, wherein the structural building blocks are precursors of inorganic silica, capable of forming 4 covalent bonds in the layer formed and the protective building blocks are organosilanes.

In one embodiment the protective layer embeds fom about 30% to about 100% of the protein e.g. the enzyme.

In one embodiment the solid carrier is selected from the group of organic particles, inorganic particles, organic-inorganic particles, self-assembled organic particles, silica particles, gold particles, magnetic particles and titanium particles.

### EXAMPLES

### Material and Methods

### Reagents:

Tetraethyl orthosilicate 99% (TEOS), (3-aminopropyl)-triethoxysilane (APTES), ammonium hydroxide (ACS grade, 28-30%), low molecular weight chitosan (50,000-190,000 Da), medium molecular weight chitosan (190,000-310,000 Da), ethanol (ACS grade, anhydrous), glutaraldehyde (grade I, 25% in water), copper sulfate, sodium ascorbate, Chelex® 100 sodium form, polysorbate 80, Sodium chloride, Recombinant Human Pancreatic lipase (HRL, certified reference material), pancreatin (4×USP specifications), bovine serum albumin (BSA), Dibenzocyclooctyne-maleimide (DBCO-mal, for copper-free click chemistry), 1,2-Di-O-lauryl-rac-glycero-3-(glutaric acid 6-methylresorufin ester), Copper sulfate pentahydrate, Sodium ascorbate, acetic acid (glacial, ACS reagent, ≥99.7%), Tris base, Casein (from bovine milk), Potassium phosphate monobasic, Potassium phosphate dibasic, Trichloroacetic acid, Sodium carbonate, Folin-Ciocalteu reagent, Amylase activity assay kit, Dimethyl sulfoxide (DMSO), MTT ((3-[4,5-dimethylthiazol-2-yl]-2,5 diphenyl tetrazolium bromide), Triton X, Alcian blue 8GX, acetic acid, Hank's Balanced Salt Solution (HBSS), Lucifer Yellow CH dilithium salt, EGTA (Ethylene glycol-bis(2-aminoethylether)-N,N,N′,N′-tetraacetic acid), PMA (Phorbol 12-myristate 13-acetate), Phalloidin-Tetramethylrhodamine B isothiocyanate, (3-Mercaptopropyl)triethoxysilane (MPTS, ≥80% GC, technical) were purchased from Sigma-Aldrich.
p-SCN-Bn-DOTA was purchased from Macrocyclics.
Benzyltriethoxysilane (B, 96%), was purchased from abcr GmbH.
3-azido-7-hydroxycoumarin was purchased from Biosynth Carbosynth.
Triethoxyethynylsilane (ETES) was purchased from Toronto Research Chemicals.
1-isothiocyanato-PEG3-Azide was purchased from BroadPharm
Silane-PEG₄-NH2 was purchased from Nanocs.
Caco-2 (human colorectal adenocarcinoma cell line) and HT29-MTX-E12 (human colon cancer cell line) was purchased from the European Collection of Authenticated Cell Cultures (ECACC).
THP-1 (human acute monocytic leukemia cell line) was purchased from LGC.
Fetal Bovine Serum, Penicillin/Streptomycin (10′000 U/ml Penicillin/10′000 g/ml Streptomycin), MEM Non-Essential Amino Acids (100×), L-Glutamine 200 mM (100×), Dulbecco's Phosphate Buffered Saline DPBS (1×), 0.25% Trypsin-EDTA (1×), RPMI 1640 Medium, DMEM, HEPES, Sodium pyruvate, D-glucose, ß-mercaptoethanol were purchased from Gibco.
Pierce™ BCA Protein Assay kit was purchased from ThermoFisher Scientific.
VECTASHIELD Antifade Mounting Medium with DAPI were purchased from Vector Laboratories.
ThinCert™ cell culture insert plates (1.0 m membrane) were purchased from Greiner bio-one.

### Synthesis Silica Nanoparticles:

Silica nanoparticles (50 nm) have been synthetized following the original Stöber process as described in WO2015/014888 A1. Briefly, ethanol, distilled water (6M) and ammonium hydroxide (0.13M) were mixed and stirred at 400 rpm for 1 h. TEOS (0.28M) was added and the solution was stirred at 400 rpm at 20° C. for 22 h. The solution was then centrifuged at 20 000 g for 20 min and washed successively with ethanol and water. Particle size measurement was carried out on SEM micrographs acquired at a magnification of 150 000× using the image analysis software Olympus stream motion.

### Enzyme Shielding and Protein Shielding:

Silica nanoparticles in water-polysorbate 80 (8 mg/L) were reacted with APTES (2.75 mM) for 30 min at 20° C. under stirring (400 rpm). Unreacted reagents were removed from the nanoparticle suspension using the amicon stirred cells with 300 kDa NMWL, Biomax polyethersulfone ultrafiltration discs (hereafter called “washing step”). These nanoparticles are further referred as amino-modified nanoparticles. Amino-modified nanoparticles were then incubated with 0.1% (v/v) of aqueous glutaraldehyde solution for 30 min at 20° C. under stirring (400 rpm). After a washing step, the nanoparticles were resuspended in MES buffer (10 mM, pH 6.2) with polysorbate 80 (8 mg/L) and reacted with enzymes (recombinant human lipase (348 μg/mL) or pancreatin (276 μg/mL)) or proteins (Bovine serum albumin, BSA) (374 μg/mL) for 1 h at 20° C. under stirring (400 rpm), further referred as immobilized-enzymes/proteins-nanoparticles. The nanoparticles were washed and resuspended in a solution of H2O-polysorbate 80 (8 mg/L) before the shielding step consisting in the polycondensation of silanes at the surface of immobilized enzymes/proteins. The different shield compositions further referred herein as “protein-shielded- and/or enzyme shielded nanoparticles” are the following:

### Shield Composed of APTES and TEOS (AT):

To the immobilized-enzymes/proteins-nanoparticles TEOS (7.75 mM) was added and allowed to react for 1 h at 20° C. under stirring (400 rpm). Subsequently, APTES (0.74 mM) was added to the reaction mixture. To obtain a full protective layer, the silane polycondensation was stopped after 21h by washing the nanoparticles suspension.

The silica nanoparticles obtained after silane polycondensation comprising serum bovine albumin protein immobilized on the surface of the silica particle which is fully embedded by the protective layer comprising polycondensed silanes have been produced as described in WO2015/014888 A1 and are further referred herein as “fully shielded nanoparticles”, “protein fully shielded nanoparticles” “Nanoparticles 1” or “NP-1”.

The silica nanoparticles obtained after silane polycondensation comprising pancreatin immobilized on the surface of the silica particle which is fully embedded by the protective layer comprising polycondensed silanes have been produced as described in WO2015/014888 A1 and are further referred herein as “fully shielded nanoparticles”, “enzyme fully shielded nanoparticles” “Nanoparticles 15” or “NP-15”.

The silica nanoparticles obtained after silane polycondensation comprising pancreatin immobilized on the surface of the silica particle which is partially embedded by the protective layer comprising polycondensed silanes have been produced as described in WO2015/014888 A1 and are further referred herein as “partially shielded nanoparticles”, “enzyme partially shielded nanoparticles” “Nanoparticles 13” or “NP-13”. To obtain the partial protective layer, the silane polycondensation was stopped after 5h by washing the nanoparticles suspension.

### Shield Composed of APTES, TEOS, and Benzyltriethoxysilane (ATB):

To the immobilized-enzymes/proteins-nanoparticles TEOS (3.87 mM) was added and allowed to react for 1 h at 20° C. under stirring (400 rpm). Subsequently, APTES (0.74 mM) and Benzyltriethoxysilane (3.34 mM) were added to the reaction mixture. The silane polycondensation was stopped after 21h by washing the nanoparticles suspension.

The silica nanoparticles obtained after silane polycondensation comprising recombinant human lipase immobilized on the surface of the silica particle which is fully embedded by the protective layer comprising polycondensed silanes have been produced as described in WO2015/014888 A1 and are further referred herein as “fully shielded nanoparticles”, “enzyme fully shielded nanoparticles” “Nanoparticles 4” or “NP-4”.

The silica nanoparticles obtained after silane polycondensation comprising pancreatin immobilized on the surface of the silica particle which is fully embedded by the protective layer comprising polycondensed silanes have been produced as described in WO2015/014888 A1 and are further referred herein as “fully shielded nanoparticles”, “enzyme fully shielded nanoparticles” “Nanoparticles 11” or “NP-11”.

Particle size measurement was carried out on SEM micrographs acquired at a magnification of 150 000× using the image analysis software Olympus stream motion.

The different nanoparticles obtained after enzymes/proteins shielding are summarized in Table 1 below.

### Labeling of Nanoparticles:

For the labeling of nanoparticles, additional steps have been added to the process of nanoparticles described in the section “Enzyme shielding and protein shielding”.

Radioactive labeling with Lutetium 177 (¹⁷⁷Lu):

All used buffers were pretreated with Chelex®.

Amino-modified nanoparticles produced according to section “Enzyme shielding and protein shielding” above were resuspended in phosphate buffer (0.1M, pH 7.4) with polysorbate 80 (8 mg/L) and p-SCN-Bn-DOTA (1 mg/mL) was added and allowed to react for 1 h at 20° C. under stirring (400 rpm). After a washing step, the DOTA-labeled-nanoparticles were resuspended in MES buffer (10 mM, pH 6.2) with polysorbate 80 (8 mg/L) and the enzyme was immobilized and shielded on these DOTA-labeled-nanoparticles as described in the section “Enzyme shielding and protein shielding” above. Then, the nanoparticles were incubated with ¹⁷⁷Lu (2500 μCi) and sodium acetate (250 mM, pH 5.4) for 12 h at 45° C. After a washing step in sodium acetate (20 mM, pH 5.0) with polysorbate 80 (8 mg/L), the nanoparticles were resuspended in EDTA (1 mM) and incubated overnight at room temperature (RT). The nanoparticles were then washed and resuspended in 0.9% sodium chloride with polysorbate 80 (8 mg/L).

Labeling with FITC:

Amino-modified nanoparticles produced according to section “Enzyme shielding and protein shielding” above were resuspended in borate buffer (50 mM, pH 8.5) with polysorbate 80 (8 mg/L) and FITC (50 μg/mL) was added and allowed to react for 1 h at 20° C. under stirring (400 rpm). After a washing step, the FITC-labeled-nanoparticles were resuspended in MES buffer (10 mM, pH 6.2) with polysorbate 80 (8 mg/L) and the enzyme was immobilized and shielded on these DOTA-labeled-nanoparticles as described in the section “Enzyme shielding and protein shielding” above.

### Surface Functionalization of the Nanoparticles:

Electrostatic Binding of Chitosan on Protein-Shielded- and/or Enzyme-Shielded Nanoparticles (Chitosan-Functionalized-Shielded-Nanoparticles)

Protein-shielded- and/or enzyme shielded nanoparticles produced according to section “Enzyme shielding and protein shielding” above in H2O with polysorbate 80 (8 mg/L) were reacted with a solution of chitosan (500 μg/mL) in 0.1M acetic acid for 30 min at 20° C. under stirring (400 rpm). After a washing step, the nanoparticles were resuspended in H2O with polysorbate 80 (8 mg/L). Full functionalization with chitosan (“fully functionalized”) was obtained by applying a chitosan concentration based on theoretical calculation of the number of anchoring points. Partial functionalization with chitosan (“partially functionalized”) was obtained by applying a fraction of this number of anchoring points (between 10% and 80%). The silica nanoparticles obtained after full functionalization with electrostatic binding of medium MW chitosan comprising BSA immobilized on the surface of the silica particle which is fully embedded by the protective layer comprising polycondensed silanes (AT) and further comprising medium MW chitosan as functional constituent immobilized on the surface of the protective layer i.e. fully functionalized with medium MW chitosan, are further referred herein as “Nanoparticles 2” or “NP-2”.

The silica nanoparticles obtained after partial functionalization with electrostatic binding of medium MW chitosan comprising BSA immobilized on the surface of the silica particle which is fully embedded by the protective layer comprising polycondensed silanes (AT) and further comprising partially medium MW chitosan as functional constituent immobilized on the surface of the protective layer i.e. partially functionalized with medium MW chitosan, are further referred herein as “Nanoparticles 3” or “NP-3”.

The silica nanoparticles obtained after full functionalization with electrostatic binding of medium MW chitosan comprising recombinant human lipase immobilized on the surface of the silica particle which is fully embedded by the protective layer comprising polycondensed silanes (ATB) and further comprising medium MW chitosan as functional constituent immobilized on the surface of the protective layer i.e. fully functionalized with medium MW chitosan, are further referred herein as “Nanoparticles 5” or “NP-5”.

The silica nanoparticles obtained after partial functionalization with electrostatic binding of medium MW chitosan comprising recombinant human lipase immobilized on the surface of the silica particle which is fully embedded by the protective layer comprising polycondensed silanes (ATB) and further comprising medium MW chitosan as functional constituent immobilized on the surface of the protective layer i.e. partially functionalized with medium MW chitosan, are further referred herein as “Nanoparticles 6” or “NP-6”.

The silica nanoparticles obtained after full functionalization with electrostatic binding of medium MW chitosan comprising pancreatin immobilized on the surface of the silica particle which is fully embedded by the protective layer comprising polycondensed silanes (ATB) and further comprising medium MW chitosan as functional constituent immobilized on the surface of the protective layer i.e. fully functionalized with medium MW chitosan, are further referred herein as “Nanoparticles 12” or “NP-12”.

The silica nanoparticles obtained after full functionalization with electrostatic binding of medium MW chitosan comprising pancreatin immobilized on the surface of the silica particle which is partially embedded by the protective layer comprising polycondensed silanes (AT) and further comprising medium MW chitosan as functional constituent immobilized on the surface of the protective layer i.e. fully functionalized with medium MW chitosan, are further referred herein as “Nanoparticles 14” or “NP-14”.

The silica nanoparticles obtained after full functionalization with electrostatic binding of medium MW chitosan comprising pancreatin immobilized on the surface of the silica particle which is fully embedded by the protective layer comprising polycondensed silanes (AT) and further comprising medium MW chitosan as functional constituent immobilized on the surface of the protective layer i.e. fully functionalized with medium MW chitosan, are further referred herein as “Nanoparticles 16” or “NP-16”.

### Click Chemistry Cu++

To a solution of protein-shielded- and/or enzyme shielded nanoparticles produced according to section “Enzyme shielding and protein shielding” above (250 μL, 10 mg/mL) was added ethyltriethoxysilane (ETES) (0.8 umol). The resulting mixture was stirred at 400 rpm, 20° C. for 15 minutes to yield ethynyl-modified nanoparticles. Then, solutions of 3-azido-7-hydroxycoumarin (0.8 μmol) in acetic acid (0.1 M), CuSO4 (83 μL, 20 mM) in H2O with polysorbate 80 (8 mg/L) and sodium ascorbate (208 μL, 100 mM) in H2O/PS80 were successively added to the nanoparticle suspension. The resulting mixture was stirred at 400 rpm, 20° C. for 22 hours. Cycloaddition reaction kinetics was monitored by collecting samples at different reaction times and measuring fluorescence (λₑₓ: 404 nm, λₑₘ: 477 nm).

### Click Chemistry Cu++Free

The pH of protein-shielded- and/or enzyme-shielded nanoparticles produced according to section “Enzyme shielding and protein shielding” above (1 mL, 10 mg/mL) was adjusted to pH 9 by adding NaOH (5 M, 1 μL). Then, a solution of dibenzocyclooctyne-maleimide (238 μL, 1 mg/mL) in DMSO was added to the nanoparticle suspension. The resulting mixture was stirred at 400 rpm, 20° C. for 30 minutes. Nanoparticles were washed three times in H2O with polysorbate 80 (8 mg/L) (1 mL) and resuspended in 1 mL of H2O with polysorbate 80 (8 mg/L) to yield dibenzocyclooctyne-modified nanoparticles (nanoparticles-DBCO).

To nanoparticles-DBCO (100 μL, 10 mg/mL) was added a solution of 3-azido-7-hydroxycoumarin (4.75 μL, 1 mg/mL) in acetic acid (0.1 M). The resulting mixture was stirred at 400 rpm, 20° C. for 6 hours. Then, nanoparticles were washed three times in H2O/PS80 (100 μL) and resuspended in 100 μL of H2O with polysorbate 80 (8 mg/L). Steady-state fluorescence measurements were performed using 100 μL of nanoparticle suspensions at 2 mg/mL (λₑₓ: 404 nm, λₑₘ: 477 nm).

Functionalization of Protein-Shielded- and/or Enzyme-Shielded Nanoparticles with Chitosan by Click Chemistry (Chitosan-Functionalized-Shielded-Nanoparticles)

The pH of protein-shielded- and/or enzyme-shielded nanoparticles produced according to section “Enzyme shielding and protein shielding” above (200 μL, 10 mg/mL) was adjusted to pH 9 by adding NaOH (5 M, 1 μL). Then, a solution of dibenzocyclooctyne-maleimide in DMSO was added to the nanoparticle suspension. The resulting mixture was stirred at 400 rpm, 20° C. for 30 minutes. The nanoparticles were washed three times in H2O with polysorbate 80 (8 mg/L) (200 μL) and resuspended in 200 μL of H2O with polysorbate 80 (8 mg/L) to yield dibenzocyclooctyne-modified nanoparticles (nanoparticles-DBCO). Then, azido-modified chitosan in acetic acid (0.1 M) was added to the nanoparticle-DBCO suspension. The resulting mixture was stirred at 400 rpm, 20° C. for 6 hours. Then, the nanoparticles were washed three times in H2O with polysorbate 80 (200 μL) and resuspended in 200 μL of H2O with polysorbate 80 (8 mg/L).

The silica nanoparticles obtained after partial functionalization with medium MW chitosan by click chemistry comprising recombinant human lipase immobilized on the surface of the silica particle which is fully embedded by the protective layer comprising polycondensed silanes (ATB) and further comprising medium MW chitosan as functional constituent immobilized on the surface of the protective layer i.e. partially functionalized with medium MW chitosan, are further referred herein as “Nanoparticles 7” or “NP-7”.

The silica nanoparticles obtained after partial functionalization with low MW chitosan by click chemistry comprising the enzyme recombinant human lipase immobilized on the surface of the silica particle which is fully embedded by the protective layer comprising polycondensed silanes (ATB) and further comprising low MW chitosan as functional constituent immobilized on the surface of the protective layer i.e. partially functionalized with low MW chitosan, are further referred herein as “Nanoparticles 8” or “NP-8”.

Functionalization of Protein-Shielded- and/or Enzyme-Shielded Nanoparticles with Silane-PEG-NH2 (Polymerized Silane-PEG-NH2-Functionalized-Shielded-Nanoparticles)

The pH of a solution of protein-shielded- and/or enzyme-shielded nanoparticles produced according to section “Enzyme shielding and protein shielding” above (2 mL, 10 mg/mL) was adjusted to pH 9 by adding NaOH (5 M). Then, Silane-PEG-NH2 (567 μL, 10 mg/mL) was added to the nanoparticle suspension. The resulting mixture was stirred at 400 rpm, 20° C. for 30 minutes. The nanoparticles were washed three times in H2O/PS80 (2 mL) and resuspended in 2 mL of H2O with polysorbate 80 (8 mg/L).

The silica nanoparticles obtained after functionalization with Silane-PEG₄-NH2 comprising the enzyme recombinant human lipase immobilized on the surface of the silica particle which is fully embedded by the protective layer comprising polycondensed silanes (ATB) and further comprising polymerized Silane-PEG₄-NH2 as functional constituent immobilized on the surface of the protective layer i.e., fully functionalized with Silane-PEG₄-NH2 are further referred herein as “Nanoparticles 10” or “NP-10”.

Functionalization of Protein-Shielded- and/or Enzyme-Shielded Nanoparticles with APTES (Polymerized APTES-Functionalized-Shielded-Nanoparticles)

The pH of a solution of protein-shielded- and/or enzyme-shielded nanoparticles produced according to section “Enzyme shielding and protein shielding” above (2 mL, 10 mg/mL) was adjusted to pH 9 by adding NaOH (5 M). Then, APTES (2.21 μL) was added to the nanoparticle suspension. The resulting mixture was stirred at 400 rpm, 20° C. for 30 minutes. The nanoparticles were washed three times in H2O with polysorbate 80 (8 mg/L) (2 mL) and resuspended in 2 mL of H2O with polysorbate 80 (8 mg/L).

The silica nanoparticles obtained after functionalization with APTES comprising recombinant human lipase immobilized on the surface of the silica particle which is fully embedded by the protective layer comprising polycondensed silanes (ATB) and further comprising polymerized APTES as functional constituent immobilized on the surface of the protective layer i.e. functionalized with polymerized APTES, are further referred herein as “Nanoparticles 9” or “NP-9”.

Functionalization of Protein-Shielded- and/or Enzyme-Shielded Nanoparticles with MPTS (Polymerized MPTS-Functionalized-Shielded-Nanoparticles)

To a solution of protein-shielded- and/or enzyme-shielded nanoparticles produced according to section “Enzyme shielding and protein shielding” above (1 mL, 10 mg/mL) in phosphate buffer (10 mM, pH 8) were added different amounts of MPTS to functionalize the nanoparticle surface with different ratios: 0.23 μmol, 0.46 μmol, 0.91 μmol, 2.3 μmol and 4.6 μmol to produce NP-17, NP-18, NP-19, NP-20 and NP-21, respectively. The resulting mixture was stirred at 400 rpm, 20° C. for 90 minutes. The nanoparticles were washed three times in H2O with polysorbate 80 (8 mg/L) (1 mL) and resuspended in 1 mL of H2O with polysorbate 80 (8 mg/L)Partial/full functionalization have been determined theoretically using a stoichiometry model calculation, which works as follows: a unit surface area corresponding to a single silanol (R—Si—OH) function on the surface of a pure silica nanoparticle has been calculated based on the silica molecule architecture. The formula to calculate the stoichiometric number of functionalities is as follows:

$$n ( - OH ) = \frac{4 m π {R}^{2}}{{V}_{U} {N}_{A} dV}$$

n(-OH): number of functionalization at the surface of the nanoparticles expressed in moles of silanol;
m: mass of the sample expressed in milligram;
R: radius of a single nanoparticle expressed in nm;
N_{A}: Avogadro number expressed in mol⁻¹;
d: density of silica nanoparticles expressed in mg·nm⁻³;
V: Volume of a single nanoparticle expressed in nm³;
V_{U}: unit surface of a single silanol function at the surface of the nanoparticle, expressed in nm².

Nanoparticles size is measured, enabling the calculation of the surface area of a single nanoparticle. Combining the unit surface area of one functional group and the surface area of the nanoparticle, the total number of available functional groups on a single nanoparticle is derived. This result is the basis for all stoichiometric calculation regarding nanoparticle functionalization.

The silica nanoparticles obtained after 5% of partial functionalization with MPTS comprising BSA immobilized on the surface of the silica particle which is fully embedded by the protective layer comprising polycondensed silanes (AT) and further comprising polymerized MPTS as functional constituent immobilized on the surface of the protective layer i.e. partially functionalized (5% of the surface of protein-shielded nanoparticles) with polymerized MPTS, are further referred herein as “Nanoparticles 17” or “NP-17”.

The silica nanoparticles obtained after 10% of partial functionalization with MPTS comprising BSA immobilized on the surface of the silica particle which is fully embedded by the protective layer comprising polycondensed silanes (AT) and further comprising polymerized MPTS as functional constituent immobilized on the surface of the protective layer i.e. partially functionalized (10% of the surface of protein-shielded nanoparticles) with polymerized MPTS, are further referred herein as “Nanoparticles 18” or “NP-18”.

The silica nanoparticles obtained after 20% of partial functionalization with MPTS comprising BSA immobilized on the surface of the silica particle which is fully embedded by the protective layer comprising polycondensed silanes (AT) and further comprising polymerized MPTS as functional constituent immobilized on the surface of the protective layer i.e. partially functionalized (20% of the surface of protein-shielded nanoparticles) with polymerized MPTS, are further referred herein as “Nanoparticles 19” or “NP-19”

The silica nanoparticles obtained after 50% of partial functionalization with MPTS comprising BSA immobilized on the surface of the silica particle which is fully embedded by the protective layer comprising polycondensed silanes (AT) and further comprising polymerized MPTS as functional constituent immobilized on the surface of the protective layer i.e. partially functionalized (50% of the surface of protein-shielded nanoparticles) with polymerized MPTS, are further referred herein as “Nanoparticles 20” or “NP-20”.

The silica nanoparticles obtained after full functionalization (100%) with MPTS comprising BSA immobilized on the surface of the silica particle which is fully embedded by the protective layer comprising polycondensed silanes (AT) and further comprising polymerized MPTS as functional constituent immobilized on the surface of the protective layer i.e. fully functionalized (100% of the surface of protein-shielded nanoparticles) with polymerized MPTS, are further referred herein as “Nanoparticles 21” or “NP-21”.

The different nanoparticles obtained and used in the Experiments outlined below are summarized in Table 1.

| | | | | Functionalization | | | | |
|---|---|---|---|---|---|---|---|---|
| | | | | Chitosan | | | Others | |
| | | | | | | Level of surface | | Level of surface |
| | | Shield | | Method of | Molecular | nanoparticles | | nanoparticles |
| Name | Bovine serum albumin | Composition | Size | binding | weight | functionalization | Description | functionalization |
| NP-1 | Bovine serum albumin | AT | 8 nm | — | — | — | — | — |
| NP-2 | Bovine serum albumin | AT | 8 nm | Electrostatic | Medium | Full | — | — |
| NP-3 | Bovine serum albumin | AT | 8 nm | Electrostatic | Medium | Partial | — | — |
| NP-4 | Recombinant human lipase | ATB | 8 nm | — | — | — | — | — |
| NP-5 | Recombinant human lipase | ATB | 8 nm | Electrostatic | Medium | Full | — | — |
| NP-6 | Recombinant human lipase | ATB | 8 nm | Electrostatic | Medium | Partial | — | — |
| NP-7 | Recombinant human lipase | ATB | 8 nm | Click chemistry | Medium | Partial | — | — |
| NP-8 | Recombinant human lipase | ATB | 8 nm | Click chemistry | Low | Partial | — | — |
| NP-9 | Recombinant human lipase | ATB | 8 nm | — | — | — | APTES | — |
| NP-10 | Recombinant human lipase | ATB | 8 nm | — | — | — | Silane-PEG-NH2 | — |
| NP-11 | Pancreatin | ATB | 8 nm | — | — | — | — | — |
| NP-12 | Pancreatin | ATB | 8 nm | Electrostatic | Medium | Full | — | — |
| NP-13 | Pancreatin | AT | 2 nm | — | — | — | — | — |
| NP-14 | Pancreatin | AT | 2 nm | Electrostatic | Medium | Full | — | — |
| NP-15 | Pancreatin | AT | 8 nm | — | — | — | — | — |
| NP-16 | Pancreatin | AT | 8 nm | Electrostatic | Medium | Full | — | — |
| NP-17 | Bovine serum albumin | AT | 8 nm | — | — | — | MPTS | 5% |
| NP-18 | Bovine serum albumin | AT | 8 nm | — | — | — | MPTS | 10% |
| NP-19 | Bovine serum albumin | AT | 8 nm | — | — | — | MPTS | 20% |
| NP-20 | Bovine serum albumin | AT | 8 nm | — | — | — | MPTS | 50% |
| NP-21 | Bovine serum albumin | AT | 8 nm | — | — | — | MPTS | 100% |

### Protein Quantification:

Immobilization yield of enzyme was quantified using the indirect Lowry protein quantification method. A standard regression curve with known concentrations of protein was build using Bovine Serum Albumin standards. The supernatant of nanoparticles after enzyme immobilization was taken and centrifuged for 3 minutes at 20 k rcf. Then, 1 mL of Lowry solution was added to 200 μL of samples and standards, vortexed and incubated at RT for 15 minutes. Subsequently, 100 μL of Folin reagent 1N were added while vortexing and incubated for 30 minutes at RT. Finally, the absorbance was read at 750 nm using Biotek Synergy H1 Reader.

### Cell Culture:

For all experiments, cells were cultured at 37° C. and 5% C02.

Caco2 (human colorectal adenocarcinoma cell line) and HT29-MTX-E12 (human colon cancer cell line) cells were cultured in DMEM supplemented with 10% heat-inactivated fetal calf serum, 2 mM L-glutamine, 1% non-essential amino-acid and 100 U/mL penicillin/streptomycin. For the development of the intestinal barrier model, cells were seeded at a density of 2.6×10⁵ cells/cm² in transwell PET inserts (1 μm pore size). All cell models were used for experiments on day 21. For the monoculture, Caco-2 cells were used. For the co-culture, Caco-2 and HT-29-MTX-E12 cells were used at a ratio 75%-25%.

THP-1 (Human monocytic leukaemia cell line) cells were maintained in culture in RPMI 1640 supplemented with 10% heat-inactivated fetal calf serum, 2 mM L-glutamine, and 100 U/mL penicillin/streptomycin.

For THP-1 differentiation into macrophages, THP-1 cells were cultured in differentiation medium: RPMI 1640 with 10% heat-inactivated fetal calf serum, 2 mM L-glutamine, 100 U/mL penicillin/streptomycin, 10 mM HEPES, 1 mM sodium pyruvate, 2.5 g/L glucose and 50pM P-mercaptoethanol. THP-1 were differentiated into M0-macrophages by 24h incubation with 150 nM phorbol 12-myristate 13-acetate (PMA) followed by 24h incubation in differentiation medium.

### Mucus Staining:

Differentiated cellular monolayers were fixed with 4% formalin for 15 min at RT followed by washing with PBS. A solution of 1% alcian blue in 3% acetic acid (pH 2.5) was added. After 30 min of incubation at RT, cells were extensively washed in PBS, and the insert membrane with cells was cut out of the plastic insert holder and mounted onto a glass slide.

### Binding of the Nanoparticles to Mucus:

For the ex-vivo evaluation of the interaction of the functionalized-shielded nanoparticles with mucus, the intestine of a freshly slaughtered pig was collected from a local abattoir. The small intestine was longitudinally incised and scrapped with a glass slide to collect the mucus. To 1 g of mucus, 5 mL of 0.1M sodium chloride was added and agitated for 1 h at 40 rpm. The suspension was then centrifuged for 2 h at 13 125 g. The clean portion of the pellet was retained, and the process was repeated once more. Transwell inserts with a surface of 33.6 mm² was covered with 50 mg of porcine mucus. The acceptor chamber was filled with 500 μL of HBSS pH 7.4. The donor chamber was filled with 250 μL of FITC-labeled nanoparticles diluted in HBSS pH 6.4. The plate was then incubated at 370 for 1 h under shaking (300 rpm). After incubation, successive washing steps were realized with H₂O, sodium chloride 0.9% and triton-X100 0.01%. The percentage of nanoparticles bound to the mucus was assessed by the measure of the fluorescence (λₑₓ: 489 nm, λₑₘ: 515 nm) in each compartment.

For the in-vitro evaluation of the interaction of the functionalized-shielded-nanoparticles with mucus, differentiated cell culture were exposed to FITC-labeled nanoparticles in DMEM for 24 h at 37° C. under shaking (300 rpm). After washing with PBS, the binding of the functionalized-shielded-nanoparticles to the mucus was evaluated optically.

### Biodistribution:

CD-1 mice were orally injected by gavage with 100 mg/kg of radioactive chitosan-functionalized-shielded-nanoparticles after overnight food fasting. At terminal time points, the animals were anesthetized by intraperitoneal injection of a mixture of ketamine hydrochloride (50 mg/kg) and xylazine hydrochloride (10 mg/kg), and then they were rapidly sacrificed by exsanguination via intracardiac puncture. The organs of interest were excised and weighed using a precision balance. From the mouse individually housed in metabolic cage, urine (including urine in bladder) and faeces have been collected at 24 hours following the administration and analysed for their radioactivity. The counting of samples radioactivity was performed in an automatic gamma counter (Wallace Wizard 2470—Perkin Elmer) calibrated for Lutetium-177 radionuclide (efficiency: 13.8%; LLOQ: 500 cpm). The radioactivity in sampled tissues was expressed as percentage of the ID per gram of tissue (% ID/g).

### Viability Assay:

Cells were plated into 96-well flat bottom cell culture plate at a density of 2×10⁴ cells/well. After 24 h, culture medium was replaced, and cells were treated with increasing concentrations of shielded- or functionalized-shielded-nanoparticles (0-1000 μg/mL) for 24 h and 48h. Cellular monolayers were rinse with medium and MTT (3-[4,5-dimethylthiazol-2-yl]-2,5-diphenyl tetrazolium bromide) solution (1 mg/mL) was added to each well. The cell cultures were incubated at 37° C. for 2 h. The formazan crystals generated during the incubation period were dissolved in DMSO. After complete solubilization, the absorbance was measured at 570 nm and 680 nm (reference wavelength) using a Synergy H1 Multimode Microplate Reader (Bio-Tek Instruments). Finally, the MTT results were expressed as a percentage of viability: [(samples absorbance−cell free sample blank)*100/(untreated samples absorbance−cell free sample blank)]. Viability of non-treated control cells was arbitrarily defined as 100%.

### Barrier Integrity:

### TEER

The integrity of the cell barrier was assessed by the measurement of the transepithelial electrical resistance (TEER) using the CellZscope system (NanoAnalytics). After cell culture medium refreshment and treatment with nanoparticles, cells were allowed to equilibrate overnight at 37° C. before TEER measurements. Inserts without cells and cellular monolayer treated with 2.5 mM of EGTA (Ethylene glycol-bis(2-aminoethylether)-N,N,N′,N′-tetraacetic acid) were used as negative control.

### Lucifer Yellow Translocation

Cell layer integrity was also assessed using Lucifer Yellow (LY) after nanoparticles exposure. The medium from Caco-2 monoculture and Caco-2/HT-29-MTX-E12 co-culture was removed and replaced by HBBS. LY (100 μM) was added apically to the cells for 90 min at 37° C. and the basolateral medium was collected and analyzed for fluorescence (λₑₓ: 428 nm; λₑₘ: 540 nm) using a Synergy H1 Multimode Microplate Reader (Bio-Tek Instruments).

### Internalization:

### Confocal

After incubation with FITC-labelled-chitosan-functionalized-shielded-nanoparticles, cell monolayers grown on filter inserts were washed in PBS and fixed in 4% formalin for 15 min at RT. Cells were washed, permeabilized with Triton 0.1% for 15 min at RT, washed three times with PBS, and blocked with BSA 1% for 30 min at RT. Subsequently, cells were incubated with phalloidin-TRITC (Tetramethyl-rhodamine B-isothiocyanate) for 1 h at RT. After a last washing step, the cells were mounted on a microscope glass slide by applying one drop of antifade mounting medium with DAPI. Fluorescence microscopic analysis were performed on a confocal laser scanning microscope (Fluoview FV3000, Olympus).

### FACS

For flow cytometry analysis, differentiated cell culture pre-treated or not with FITC-labelled-chitosan-functionalized-shielded-nanoparticles were detached with trypsin and washed with PBS. Cells were then processed for data acquisition using the flow cytometer Cell Sorter SH800Z (Sony). Data were analysed using Flowjo 10 software.

### Human Recombinant Lipase Activity Assay:

To a solution of shielded-nanoparticles comprising lipase and chitosan-functionalized-shielded-nanoparticles comprising lipase (60 μL, 3.3 mg/mL) in Tris buffer (0.1 M, pH 8.4) was added 1,2-Di-O-lauryl-rac-glycero-3-(glutaric acid 6-methylresorufin ester) (60 μL, 100 pM). Lipase activity kinetics was monitored by steady-state fluorescence measurement (λex/λem=529/600 nm) in a dark 96-well plate for 30 min at 37° C.

Lipase from Pancreatin Activity Assay:

To a solution of shielded-nanoparticles comprising pancreatin and chitosan-functionalized-shielded-nanoparticles comprising pancreatin (125 μL, 3.33 mg/mL) in Tris buffer (0.1 M, pH 8.4) was added 1,2-Di-O-lauryl-rac-glycero-3-(glutaric acid 6-methylresorufin ester) (125 μL, 50 μM). The resulting mixture was stirred at 37° C., 750 rpm for 30 minutes in the dark. The nanoparticles were centrifuged and the supernatant containing the reaction products was collected. Steady-state fluorescence measurement (λex/λem=529/600 nm) was measured in a dark 96-well plate, on 100 μL of the resulting solutions.

### Protease Activity Assay:

To a solution of shielded-nanoparticles comprising pancreatin and chitosan-functionalized-shielded-nanoparticles comprising pancreatin (50 μL, 10 mg/mL) in phosphate buffer (50 mM, pH 7) was added a solution of casein (250 μL, 0.65% w/v) in phosphate buffer (50 mM, pH 7). The resulting mixture was stirred at 37° C., 750 rpm for 30 minutes. The nanoparticles were centrifuged and the supernatant containing the reaction products was collected. To the supernatant (200 μL) was added a solution of TCA (110 mM, 167 μL) in H2O, leading to the precipitation of the undigested casein. The resulting mixture was stirred at 37° C., 750 rpm for 30 minutes. The resulting solution was centrifuged and the supernatant containing the digested fragments of casein was collected. To this supernatant (200 μL) was added a solution of sodium carbonate (500 mM, 500 μL) followed by Folin-Ciocalteu reagent (0.5 M, 100 μL). The resulting mixture was stirred at 37° C., 750 rpm for 30 minutes. Absorbance was measured at 660 nm, on 200 μL of the resulting solutions.

### Protease Activity Assay in Acidic Conditions:

Chitosan was coated at the surface of shielded-nanoparticles comprising pancreatin in acetic acid (0.1 M, pH 4). To assess the effect of chitosan on protease stability, the shielded-nanoparticles comprising pancreatin was incubated in acetic acid (0.1 M, pH 4) for 30 minutes. The activities of both shielded-nanoparticles comprising pancreatin and chitosan-functionalized-shielded-nanoparticles comprising pancreatin after incubation in acetic acid (0.1 M, pH 4) were compared to the shielded-nanoparticles comprising pancreatin activity in basic pH (pH 8).

### Amylase Activity Assay:

To a solution of chitosan-functionalized-shielded-nanoparticles comprising pancreatin (2 μL, 10 mg/mL) in activity buffer (51 μL) was added a solution of 4-Nitrophenyl-4,6-Ethylidene-a-D-Maltoheptaoside in activity buffer (107 μL). The resulting mixture was stirred at 25° C., 750 rpm for 15 minutes. The nanoparticles were centrifuged and the supernatant containing the reaction products was collected. Absorbance was measured at 405 nm, on 150 μL of the resulting solutions.

### Results

### Example 1: Surface Functionalization of the Nanoparticle with Chitosan (Comparative Experiment)

To generate mucoadhesive nanoparticle, the surface of the protective layer of shielded nanoparticles produced as described in WO2015/014888 A1 was functionalised with chitosan.

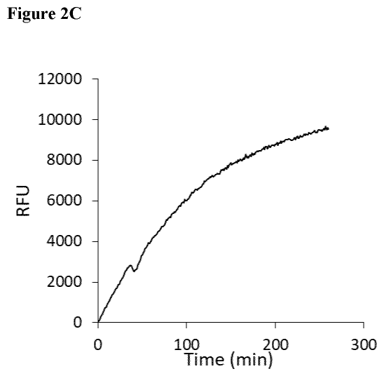

Different surface modification strategies were evaluated: non-covalent and covalent conjugation. The electrostatic binding of chitosan on shielded nanoparticles (NP-1) has been assessed using FITC-labelled chitosan. The increase of fluorescence after the conjugation on reacted nanoparticles (NP-2) compared to shielded nanoparticles (NP-1) (6632 vs 2556 respectively) demonstrates the binding of FITC-chitosan at the surface of nanoparticles (FIG. 2A). The covalent conjugation of chitosan on NP-1 has been performed by Copper II-free- and Copper II-dependent-click chemistry. To validate the binding strategy, an azido-fluorescent dye has been used, the 3-Azido-7-hydroxycoumarin. The increase of fluorescent on reacted nanoparticles demonstrates the functionalization of the shielded nanoparticles using both click reactions (FIG. 2B-C). An azido-chitosan (N₃-Chitosan) has been synthetized to proceed to its covalent binding at the surface of shielded nanoparticles. These results validate the strategies to functionalize nanoparticles with chitosan.

### Example 2: Ex-Vivo Interaction of Chitosan-Functionalized Nanoparticles with Mucus Layer: Impact of the Level of Surface Functionalization of the Nanoparticle. (Comparative Experiment)

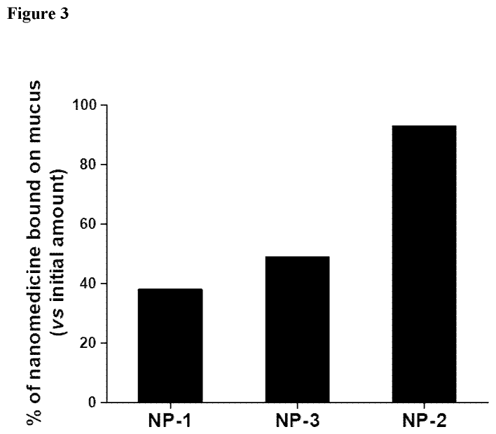

As functionalized nanoparticles have to interact with the mucus, an ex-vivo testing model has been set-up by adding a layer of porcine intestinal mucus into a transwell insert. Mucus binding studies have been then performed with fluorescent nanoparticles exhibiting different level of functionalization, either a full coverage (NP-2) or a 10% coverage (NP-3) (partial functionalization) of the nanoparticles surface with chitosan. Compared to the non-functionalized nanoparticles (NP-1), the presence of chitosan at the surface of the nanoparticles increases the interactions with the mucus. Moreover, FIG. 3 shows that the nanoparticles fully functionalized (NP-2) with chitosan interacts at a higher extend than the one with only 10% of surface functionalization (NP-3) (93% vs 49% of the applied amount respectively). Non-functionalized nanoparticles (NP-1) show only a weak interaction. These results demonstrate the mucoadhesive properties of chitosan-functionalized nanoparticles and highlight that the level of interaction of the nanoparticles with the mucus can be modulated by tuning the degree of surface functionalization.

### Example 3: Ex-Vivo Interaction of Chitosan-Functionalized Nanoparticles with Mucus Layer: Process of Immobilization and Size of Chitosan. (Comparative Experiment)

Chitosan at the surface of nanoparticles favours their interaction with the mucus. The impact of the process of chitosan immobilization and the size of the sugar on the interaction with the mucus were assessed.

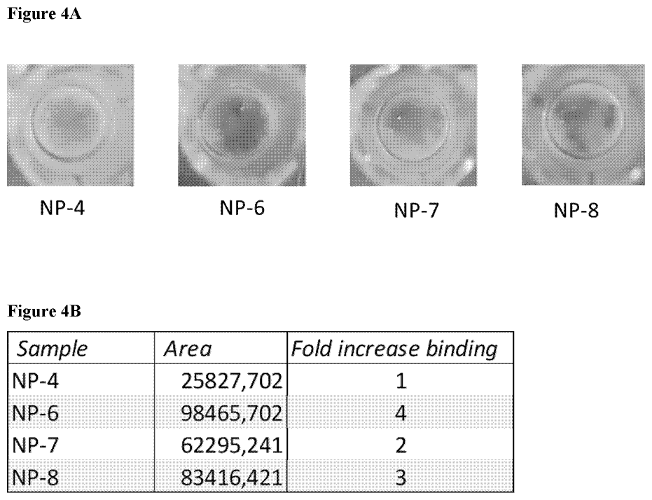

Different strategies of chitosan immobilization were evaluated: electrostatic interactions (non-covalent) and click-chemistry (covalent). FIG. 4 demonstrates that nanoparticles modified by electrostatic binding of chitosan (NP-6) and by click-chemistry (NP-7 and NP-8) interact with the mucus layer, whereas non-functionalized nanoparticles (NP-1) show only a weak interaction. It shows that both strategies can be used to functionalize the nanoparticles without impacting their biological behaviour.

Moreover, chitosan with variable molecular weights (low: 50,000-190,000 Da (NP-8) or medium: 190,000-310,000 Da (NP-7)) were immobilized at the surface of the nanoparticles by click chemistry. Results shows that the interaction of the nanoparticles with the mucus is maintained in both cases. Differences of retention of nanoparticles on the mucus between NP-7 and NP-8 can be explained by the amount of sugar residues immobilized at the surface (23 chitosan on NP-7 vs 47 chitosan on NP-8).

### Example 4: Ex-Vivo Interaction of Nanoparticles Modified with Different Polymer Comprising Amino Groups with Mucus Layer

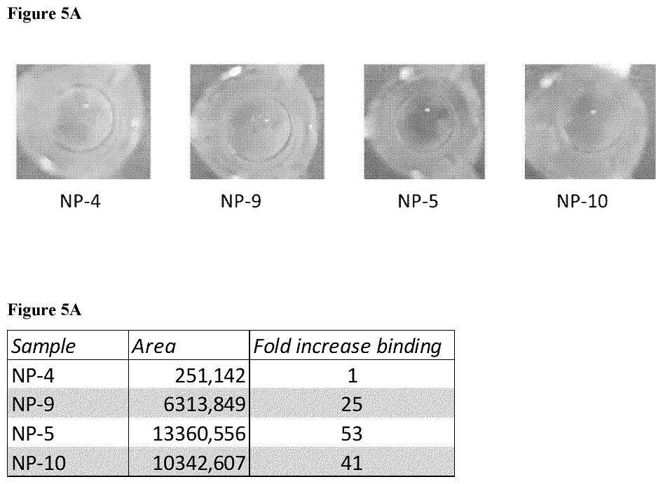

We hypothesized that the interactions of functionalized nanoparticles with mucus was not only due to chitosan but more generally to polymer comprising amino groups. To evaluate this hypothesis, shielded nanoparticles with different functional groups were generated. Ex-vivo mucus binding studies were performed with shielded nanoparticles exhibiting various polymer comprising amino groups and their interactions with the mucus layer were quantified with ImageJ software. FIG. 5 shows that the surface functionalization of the shielded nanoparticles with all polymers comprising amino groups (NP-9: addition of a layer of polymerized APTES at the surface; NP-5: surface functionalization with chitosan and NP-10: surface functionalization with silane-PEG4-NH2) used surprisingly increases the retention of nanoparticles on the mucus layer.

Altogether, these results highlight the major role of polymer comprising amino groups in the process of nanoparticles interactions with the mucus.

### Example 5: In Vitro Interaction of Chitosan-Functionalized Nanoparticles with an Intestinal Barrier Model

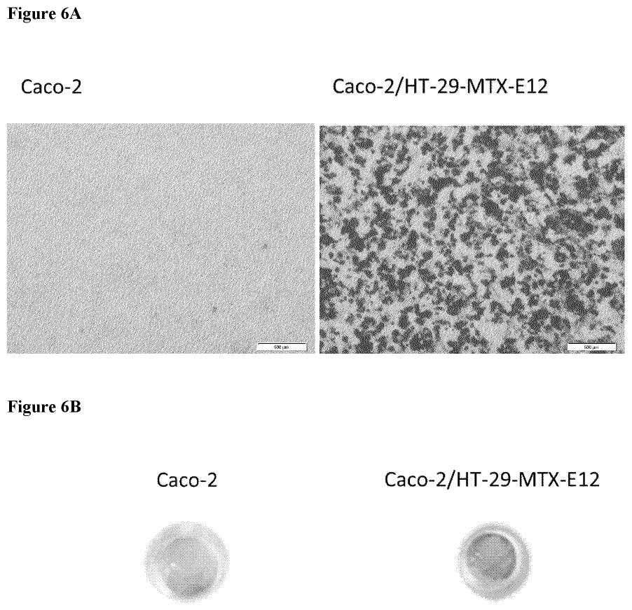

In order to confirm the interaction of chitosan-functionalized nanoparticles with the intestinal mucus, in vitro models of intestinal barriers have been developed: a non-mucus producing barrier (Caco-2 monoculture) and a mucus producing barrier (Caco-2/HT29-MTX-E12 co-culture). To visualize the mucus on top of cell layer surface, differentiated cells were stained with Alcian blue. The presence of mucus in blue was detected on the co-culture cell layer but not on the monoculture (FIG. 6A). Both intestinal barrier models were apically exposed to fluorescent chitosan-functionalized shielded nanoparticles (NP-2) for 24 h and the interaction of the nanoparticles with the barriers were optically observed. FIG. 6B shows that the presence of mucus increases the retention of NP-2 at the surface of the co-culture barrier compared to the monoculture. These results demonstrate the specific mucus targeting of chitosan-functionalized nanoparticles and confirm the mucoadhesive properties of these nanoparticles.

### Example 6: Biodistribution of Chitosan-Functionalized Nanoparticles in Mice (Comparative Experiment.)

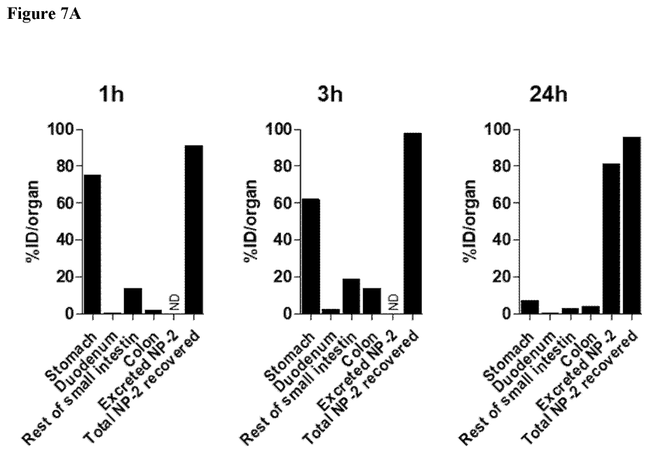

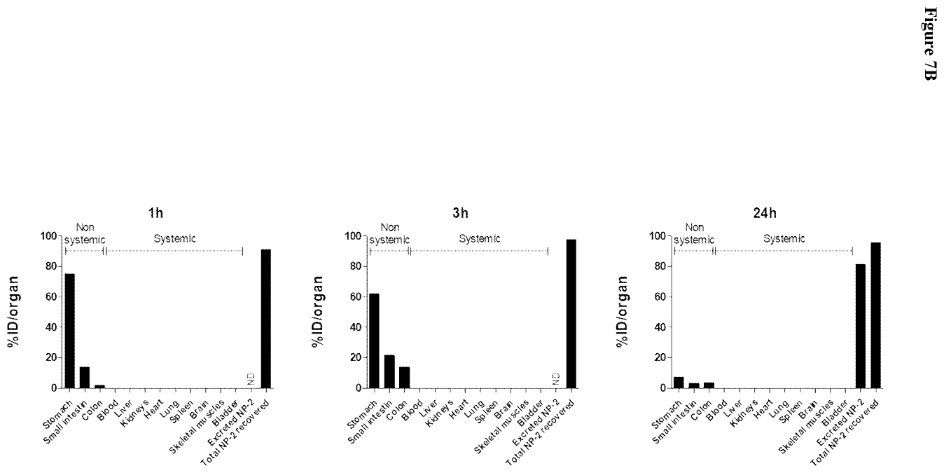

To evaluate the ability of the nanoparticles to interact with the gastrointestinal mucus in vivo, radiolabelled chitosan-functionalized shielded nanoparticles (NP-2) were orally administrated to CD-1 mice by gavage. At different time points (1 to 24h), tissues, blood, faeces and urine radioactivity were performed. The distribution of the nanoparticles (NP-2) in the digestive system shows a transit of NP-2 in the gastrointestinal tract. Compared to the residence time of the food bolus in mice stomach (1 h), the retention time of the chitosan-functionalized nanoparticles is increased: 60% of the initial amount of NP-2 remains in the stomach 3h after gavage (FIG. 7A). Moreover, the excretion balance has been followed by sampling urine and faeces of a mouse housed in metabolic cage. At 24 h, 95% of the initial amount of administrated nanoparticles was recovered, which correspond to the turnover time of the intestinal mucus in mice (FIG. 7A). These results demonstrate the surprising mucoadhesive and specific distribution properties of the chitosan-functionalized shielded nanoparticles in vivo.

To ensure the specific localization of NP-2 in the digestive system, a biodistribution on whole organism has been performed at different time points (1 to 24h). Results shows that orally administrated NP-2 remains in the gastrointestinal tract and do not cross the intestinal barrier as no radioactive signal has been detected at the systemic level (FIG. 7B). This result shows an unexpected high degree of safety of NP-2 and suggests that the nanoparticles do not disrupt the intestinal barrier integrity.

Altogether, these in vivo data reveals that the surface functionalization of the shielded nanoparticles with chitosan allows a specific targeting of the intestinal mucus, suggesting the possibility to temporary engraft the nanoparticle onto the intestinal wall without any toxicity.

### Example 7: In Vitro Cytotoxicity Assessment of the Nanoparticles (Comparative Experiment)

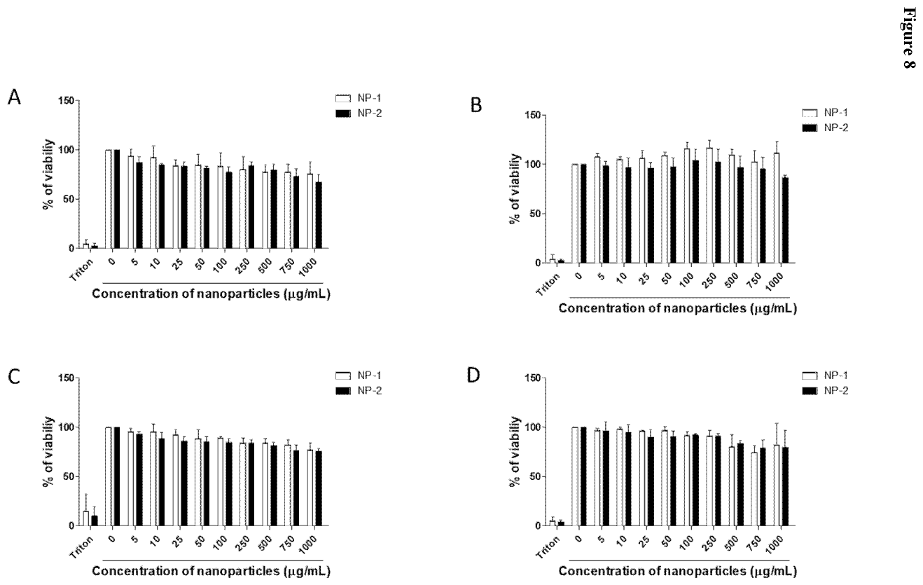

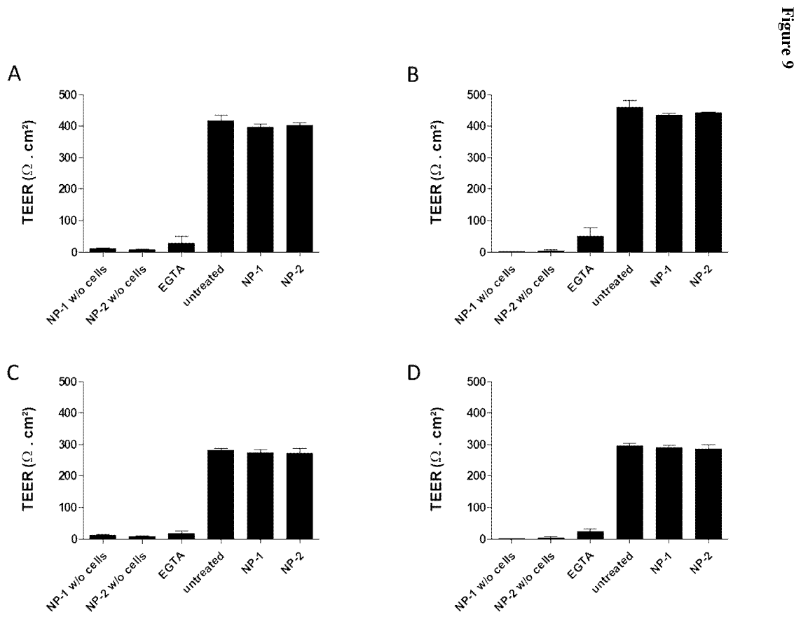

In order to evaluate the safety of the nanoparticles, human colon adenocarcinoma cells Caco-2 (FIG. 8A-B) and HT-29-MTX-E12 (FIG. 8C-D) were treated with increasing concentrations of non-functionalized- (NP-1) and chitosan-functionalized- (NP-2) shielded nanoparticles (5 to 1000 mg/mL) for 24h (FIG. 8A-C) and 48h (FIG. 8B-D). Cell viability was assessed by the measurement of the activity of the mitochondrial dehydrogenase with an MTT assay. The results show that both nanoparticles do not impact significantly the cellular viability supporting the biocompatibility of the nanoparticles with intestinal epithelial cells.

### Example 8: Effects of Nanoparticles Exposure on the Transepithelial Electrical Resistance (TEER) (Comparative Experiment)

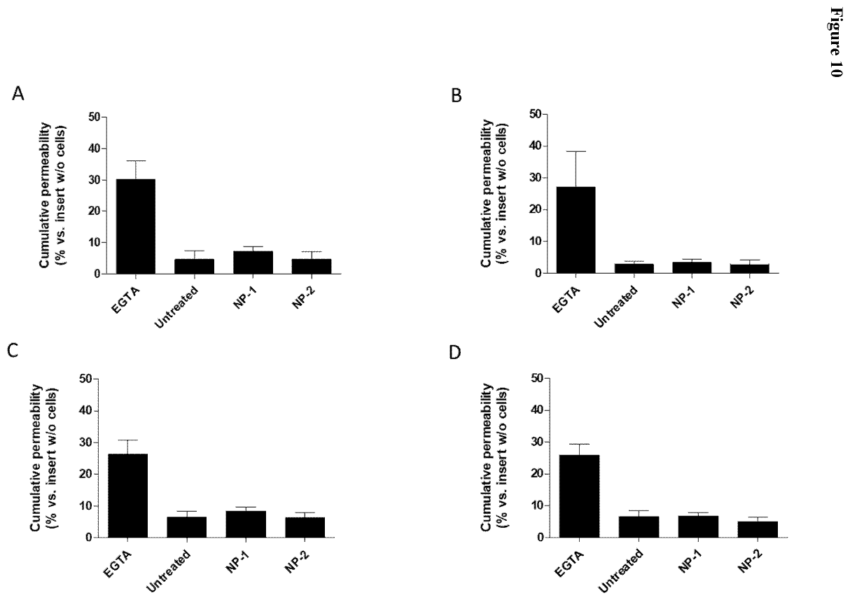

The transepithelial electrical resistance (TEER) value is a parameter commonly used to monitor the integrity and viability of cell monolayer. On day 21, differentiated Caco-2- (FIG. 8A-B) and Caco-2/HT-29-MTX-E12- (FIG. 8C-D) monolayers were apically exposed to non-functionalized (NP-1) and chitosan-functionalized shielded nanoparticles (NP-2) for 24h (FIG. 9A-C) and 48h (FIG. 9B-D), and TEER values were recorded using the CellZscope system. The opening of the tight junctions with EGTA induced a decrease of the TEER values compared to the untreated monolayer. FIG. 6 shows that exposure to the nanoparticles do not affect the TEER values of the mono- or co-culture compared to the untreated monolayers. These results demonstrate the safety and biocompatibility of the chitosan-functionalized shielded nanoparticles with the model of intestinal barrier.

### Example 9: Effects of Nanoparticles Exposure on the Translocation of Lucifer Yellow Through the Intestinal Barrier

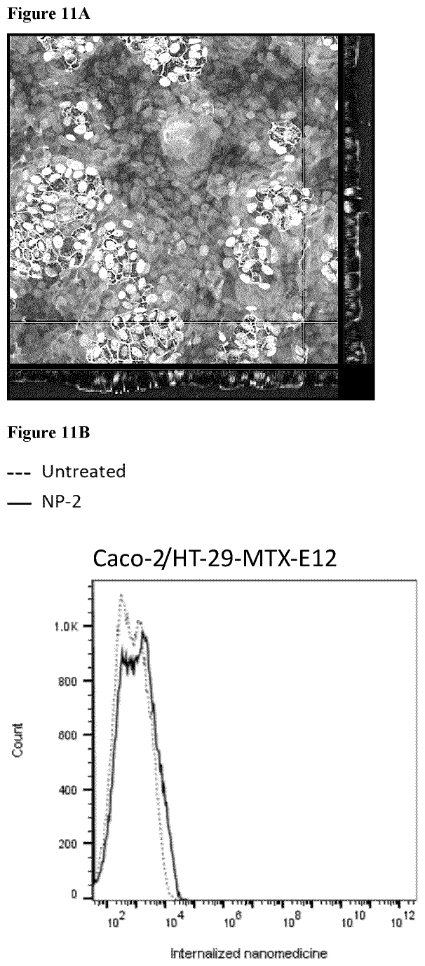

To confirm the absence of intestinal barrier disruption by the nanoparticles, translocation studies have been assessed using lucifer yellow (LY), a marker of barrier integrity. On day 21, differentiated Caco-2- (FIG. 10A-B) and Caco-2/HT-29-MTX-E12- (FIG. 10C-D) monolayers were apically exposed to non-functionalized (NP-1) and chitosan-functionalized shielded nanoparticles (NP-2) for 24h (FIG. 10A-C) and 48h (FIG. 10B-D). Then, the leakage of LY in the basolateral compartment have been measured over a period of 90 min. Results shows that the opening of the tight junction with EGTA increases the permeability of the LY, whereas the permeability of the fluorescent dye remains constant upon exposure to the nanoparticles in both models. This translocation study validates the safety and biocompatibility of the chitosan-functionalized shielded nanoparticles with the model of intestinal barrier.

### Example 10: Evaluation of the Cellular Uptake of the Nanoparticles in the Intestinal Barrier Model

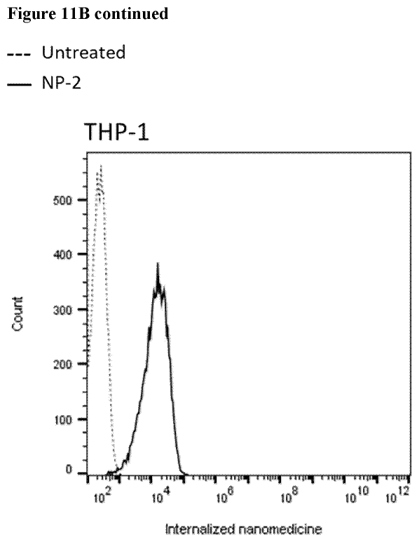

To further evaluate potential toxicity and risk assessment, the cellular uptake of the nanoparticles has been evaluated by confocal microscopy and flow cytometry after 24h exposure of the co-culture monolayer with fluorescent-NP-2. As observed on three-dimensional (3D) reconstitution z-slides obtained by confocal microscopy, the nanoparticles were found only at the surface of Caco-2/HT-29-MTX-E12 co-culture (FIG. 11A). The chitosan-functionalized shielded nanoparticles form a coating on top of the cells that can be explained by the interaction of NP-2 with the mucus. The absence of cellular uptake was confirmed by flow cytometry. The fluorescence of intestinal cells exposed to fluorescent NP-2 was not increased compared to untreated cells, contrary to the fluorescence of THP-1 differentiated into macrophages used as positive control (FIG. 11B). Altogether, these data demonstrate the safety of chitosan-functionalized shielded nanoparticles on the intestinal barrier.

### Example 11: Stabilization of the Activity of Pancreatin Immobilized and Protected on Nanoparticles

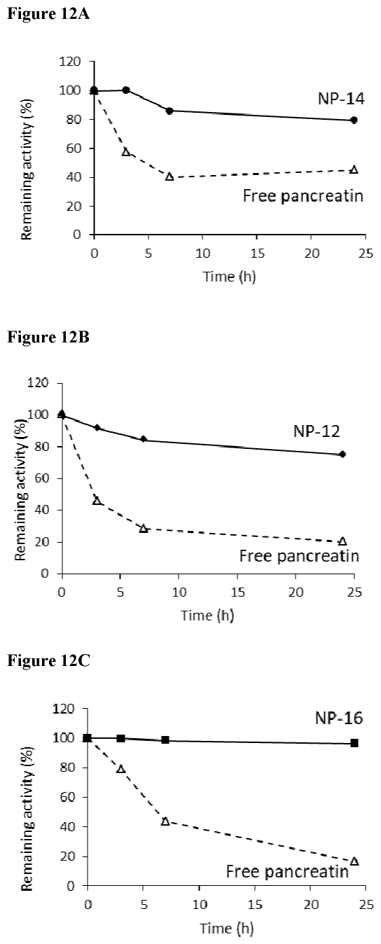

The biocatalytic activity of immobilized and protected pancreatin was assessed and compared to the free pancreatin for 24 h at 37° C. The results as displayed in FIG. 12 show an increase of the half-life of the protease (A), lipase (B) and amylase (c) activities of respectively NP-14, NP-12, and NP-16, compared to the free pancreatin. Theses result highlights the benefits of the protective layer of organosilanes on the stabilization of the biocatalytic activity of NP-12, NP-14, and NP-16.

### Example 12: Protective Effect of Chitosan on Protease Activity

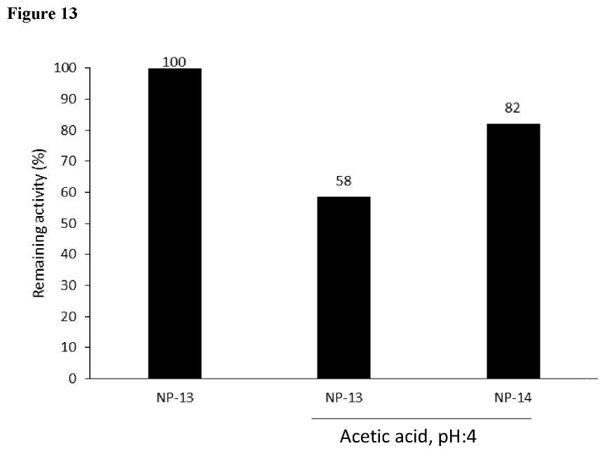

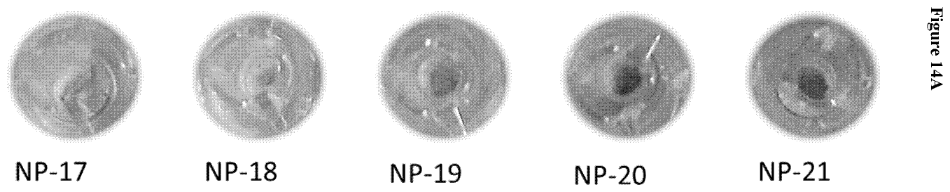

In order to evaluate the lipase activity of pancreatin shielded nanoparticles functionalized or not with chitosan in an environment close to the digestive track, we assessed a lipase activity assay in acidic conditions, at pH 4. Unexpectedly, FIG. 13 shows a higher lipase activity on chitosan functionalized shielded pancreatin nanoparticles (NP-14) compared to the unfunctionalized shielded pancreatin nanoparticles (NP-13) (82% vs 58% of remaining activity respectively) in acidic conditions. This result demonstrates that additionally to its mucoadhesive properties, chitosan exerts an unexpected protective effect of the immobilized enzymes.

### Example 13: Ex-Vivo Interaction of Nanoparticles Functionalized with Various Ratio of Thiol Group Comprising Compounds with Mucus Layer

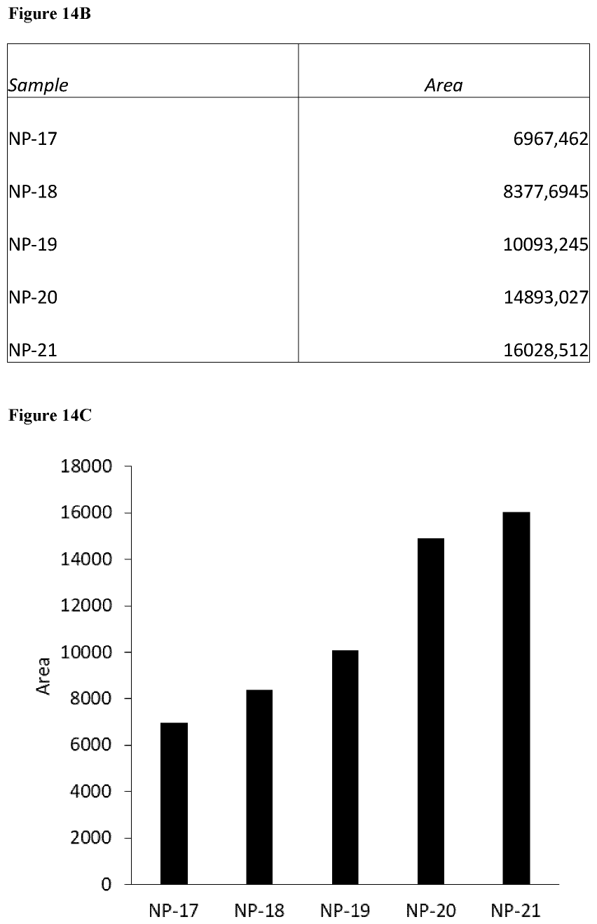

We hypothesized that the interactions of functionalized nanoparticles with mucus could also be triggered by thiol function. To evaluate this hypothesis, shielded nanoparticles with increasing ratio of thiol functions at the surface of the nanoparticles were generated according to section “Functionalization of protein-shielded- and/or enzyme-shielded nanoparticles with MPTS (Polymerized MPTS-functionalized-shielded-nanoparticles)” above. Ex-vivo mucus binding studies were performed with shielded nanoparticles exhibiting various ratio of thiol functions at their surface and their interactions with the mucus layer were quantified with ImageJ software. FIG. 14 shows that the level of interaction of the nanoparticles with the mucus layer is increasing with the amount of thiol functions at the surface of the nanoparticles.

These results demonstrate that nanoparticles functionalized with various ratio of thiol function group comprising polymers show a concentration depending binding to mucus, thereby making this functionalized nanoparticles feasible for therapeutic applications such as ERT.

1. A composition comprising a solid carrier, a protein or a fragment thereof immobilized on the surface of the solid carrier, a protective layer to protect the protein or the fragment thereof by embedding the protein or the fragment thereof, and a functional constituent immobilized on the surface of the protective layer, wherein the functional constituent immobilized on the surface of the protective layer is a polymer comprising repeat units wherein each repeat unit comprises at least one amino group and/or at least one thiol group.

2. The composition according to claim 1, wherein the polymer comprising repeat units wherein each repeat unit comprises at least one amino group and/or at least one thiol group is selected from the group consisting of a polyglucosamin, a polymerized silane-PEG-NH2 and a polymerized silane comprising an amino group.

3. The composition according to claim 1, wherein the polymer comprising repeat units wherein each repeat unit comprises at least one amino group and/or at least one thiol group is selected from the group consisting of a polyglucosamin, a polymerized silane-PEG-NH2 and a polymerized APTES.

4. The composition according to anyone of claims 1-3, wherein the polymer comprising repeat units wherein each repeat unit comprises at least one amino group and/or at least one thiol group is a polyglucosamin selected from the group consisting of chitin, chitosan, polyglucosaminoglycans, chondroitin, heparin, keratan and dermatan or a derivative thereof.

5. The composition according to claim 1, wherein the polymer comprising repeat units wherein each repeat unit comprises at least one amino group and/or at least one thiol group is selected from the group consisting of a polymerized silane comprising a thiol group, a polycarbophil-cysteine conjugate, a polymerized silane-PEG-thiol and a polycysteine.

6. The composition according to claim 1, wherein the polymer comprising repeat units wherein each repeat unit comprises at least one amino group and/or at least one thiol group is polymerized MPTS.

7. The composition according to anyone of claims 1-6, wherein the functional constituent is immobilized on the surface of the protective layer by non-covalent binding or by covalent binding.

8. The composition according to anyone of claims 1-7, wherein the protein or a fragment thereof is an enzyme or a fragment thereof.

9. The composition according to anyone of claims 1-8, wherein the protein or a fragment thereof is selected from the group consisting of serum albumin or a fragment thereof, lipase or a fragment thereof, pancreatin and a protein or fragment thereof comprised by pancreatin.

10. The composition according to anyone of claims 1-8, wherein the protein or a fragment thereof is selected from the group consisting of serum albumin or a fragment thereof, lipase or a fragment thereof, and pancreatin.

11. The composition according to anyone of claims 1-8, wherein the protein or a fragment thereof is lipase or a fragment thereof.

12. The composition of anyone of claims 1-11, wherein the protective layer embeds the solid carrier and embeds the protein or a fragment thereof immobilized on the surface of the solid carrier.

13. The composition of anyone of claims 1-112, wherein the functional constituent immobilized on the surface of the protective layer is not embedded by the protective layer.

14. The composition according to anyone of claims 1-13, wherein the composition further comprises a chelating agent, wherein the chelating agent optionally comprises a radioactive or luminescent label.

15. The composition of anyone of claims 1-14, for use as a medicament.

16. The composition of anyone of claims 1-14, for use in a method of enzyme replacement therapy (ERT) preferably gastrointestinal enzyme replacement therapy, or for use in a method for the prevention, delay of progression or treatment of exocrine pancreatic insufficiency (EPI), lactase deficiency, sucrase-isomaltase deficiency, disaccharidoses intolerances, peptides allergies, inflammatory bowel disease (IBD), cystic fibrosis, a lung disease or disorder selected from the group consisting of Gaucher, Fabry and mucopolysaccharidosis (MPS).

17. A method of producing a composition, the composition comprising a solid carrier, protein or a fragment thereof immobilized on the surface of the solid carrier, a protective layer to protect the protein or the fragment thereof by embedding the protein or the fragment thereof, and a functional constituent immobilized on the surface of the protective layer, wherein the functional constituent immobilized on the surface of the protective layer is a polymer comprising repeat units, wherein each repeat unit comprises at least one amino group and/or at least one thiol group, the method comprising the following steps:
(a) providing a solid carrier;
(b) immobilizing a protein or a fragment thereof on the solid carrier;
(c) forming a protective layer on the surface of the solid carrier to protect the protein or the fragment thereof immobilized on the solid carrier;
(d) immobilizing a functional constituent on the surface of the protective layer, wherein the functional constituent immobilized on the surface of the protective layer is a polymer comprising repeat units, wherein each repeat unit comprises at least one amino group and/or at least one thiol group.
